(19) United States
(12) Patent Application Publication
(10) Pub. No.: US 2022/0273584 A1
(43) Pub. Date: Sep. 1, 2022

(54) Film Formulation Comprising Carriers

(71) Applicant: Klaria Pharma Holding AB, Uppsala (SE)

(72) Inventors: Scott Boyer, Uppsala (SE); Fredrik Hübinette, Uppsala (SE)

(21) Appl. No.: 17/632,599

(22) PCT Filed: Aug. 14, 2020
(86) PCT No.: PCT/EP2020/072894

§ 371 Date: Feb. 3, 2022

(30) Foreign Application Priority Data
Aug. 15, 2019 (GB) 1911715.9

(51) Int. Cl. A61K 9/70; A61K 31/7036; A61K 45/06; A61K 9/127; A61P 31/04; A61K 9/00

(52) U.S. Cl. CPC A61K 9/7007; A61K 31/7036; A61K 45/06; A61K 9/1271; A61P 31/04; A61K 9/006

(57) ABSTRACT

The present invention relates to films comprising an alginate salt of a monovalent cation or a mixture of alginate salts containing at least one alginate salt of a monovalent cation, and a carrier system comprising (a) a carrier, (b) a pathogen entry protein or fragment thereof, which specifically binds to a molecule on the surface of a mammalian target cell of said pathogen and which is covalently linked to the surface of said carrier, and (c) at least one active pharmaceutical ingredient. The present invention further relates to methods for manufacturing such films, and the use of such films in the treatment of a human patient.

### FIELD OF THE INVENTION

The present invention relates to films comprising an alginate salt of a monovalent cation or a mixture of alginate salts containing at least one alginate salt of a monovalent cation, and a carrier system comprising (a) a carrier, (b) a pathogen entry protein or fragment thereof, which specifically binds to a molecule on the surface of a mammalian target cell of said pathogen and which is covalently linked to the surface of said carrier, and (c) at least one active pharmaceutical ingredient. The present invention further relates to methods for manufacturing such films, and the use of such films in the treatment of a human patient.

### BACKGROUND TO THE INVENTION

Liposomes mimic natural cell membranes and have long been investigated as drug carriers due to excellent entrapment capacity, biocompatibility and safety. They typically possess low toxicity, and are able to entrap water-soluble pharmacological agents in their internal aqueous compartment or inter-bilayer spaces (if they are multilamellar), and water-insoluble agents within their lipid membrane(s). They also provide the protection for the encapsulated pharmacological agents from the external environment.

In recent years, the idea of using bacterial surface protein invasin in targeted oral drug delivery has been considered. Invasin was used to mediate gene delivery, where a fragment of invasin was attached to non-specific DNA-binding domains (SPK). This complex was able to bind β_{ι}-integrin receptors. Approaches attaching peptide tags on nanoparticles to initiate or enhance nanoparticles uptake by mammalian cells have significantly increased over the past years. Invasin-decorated carriers were found to be useful as a “bacteriomimetic” delivery system, successfully mimicking invasive bacteria expressing internalization factors integrated in the outer membrane of their cell envelope. This enables the production of a carrier system which could enhance the cellular permeability of hydrophilic drugs for treatment of infectious disease, but with reduced toxicity due to encapsulation into nanoparticles. [1]

Liposomes have been formulated as solutions, aerosols, in a semisolid form or dry vesicular powder (pro-liposomes for reconstitution). However, despite the high success of liposomes that are delivered parentally, oral delivery of liposomes is impeded by various barriers such as instability, poor permeability across the gastrointestinal (GI) tract and difficulties with mass production, such as large batch-to-batch variations. [2]

Thus, there is a need to develop formulations of carrier systems, such as liposomes containing an active agent and bound to a targeting protein such as a pathogen entry protein, which can be administered in a non-invasive fashion, is needle-free and which is also stable and/or results in acceptable bioavailability of the active agent, preferably with low dose variability between patients.

### SUMMARY OF THE INVENTION

The present invention is based on the unexpected finding that formulations of a carrier system, comprising a carrier, a pathogen entry protein and an active pharmaceutical agent or a pharmaceutically acceptable salt thereof, in a film suitable for administration to an oral cavity can provide an advantageous balance of properties. In particular, these film formulations can potentially provide a more convenient administration than parenteral formulations. Further, the formulations may also be stable during storage and/or enabling acceptable plasma levels of active agent to be delivered to patients and/or providing low variability between patients. This therefore makes the present film formulations attractive alternatives to oral formulations.

Hence, the invention provides for the first time a film suitable for administration to an oral cavity comprising:
(i) an alginate salt of a monovalent cation or a mixture of alginate salts containing at least one alginate salt of a monovalent cation; and
(ii) a carrier system comprising:
   (a) a carrier,
   (b) a pathogen entry protein or fragment thereof, which specifically binds to a molecule on the surface of a mammalian target cell of said pathogen and which is covalently linked to the surface of said carrier, and
   (c) at least one active pharmaceutical ingredient (API) or pharmaceutically acceptable salt thereof.

In another aspect, the present invention provides a film according to the invention for use in the treatment of a human patient.

In another aspect, the present invention provides a film according to the invention for use in the treatment or prophylaxis of a disease or condition selected from: infectious diseases, preferably systemic infection; diabetes mellitus; insulinoma, metabolic syndrome; and polycysic ovary syndrome.

In a further aspect, the present invention provides a method of treating a disease or condition selected from infectious disease, diabetes mellitus, insulinoma, metabolic syndrome and polycysic ovary syndrome in a human patient, wherein said method comprises administration of at least one film according to the invention to a human patient.

In another aspect, the present invention provides the use of a film according to the invention for the manufacture of a medicament for treating a disease or condition selected from infectious disease, diabetes mellitus, insulinoma, metabolic syndrome and polycysic ovary syndrome in a human patient.

In another aspect, the present invention provides a method of manufacturing a film according to the invention, said method comprising the following steps:
(a) covalently linking a pathogen entry protein or part thereof to a carrier either prior or after contacting the carrier with at least one API or a pharmaceutically acceptable salt thereof, to form a carrier system;
(b) either the steps of:
   (i) mixing the carrier in water, and optionally subsequently adjusting the pH of the solution to the desired level by addition of an appropriate acid or base, preferably a concentrated acid, and preferably adjusting the pH of the solution to from 2 to 4;
   (ii) optionally, mixing one or more excipients into the solution; and
   (iii) adding the alginate salt of monovalent cation under suitable conditions to result in the formation of a viscous cast;
   or alternatively the steps of:
   (i) mixing the carrier in an oil phase;
   (ii) premixing a surfactant and a cosolvent, and then adding this to the solution obtained;
   (iii) optionally, adding one or more excipients, flavouring agents, buffering components, permeation enhancers, chelating agents, antioxidants and/or antimicrobial agents to water in step (i) under mixing;
   (iv) adding water, or the solution obtained in step (iii), to the solution obtained in step (ii) under stirring, preferably continuous stirring, and more preferably wherein the water or the solution obtained in step (iii) is added in a dropwise fashion; and
   (v) mixing the alginate salt of monovalent cation in the solution, until a lump free dispersion is achieved, and optionally adding further water to modulate the viscosity of the cast formed;
(c) adjusting the pH of the solution to the desired level by addition of an appropriate acid or base, preferably a diluted acid or alkali, and preferably adjusting the pH of the solution to from 3 to 5;
(d) optionally, sonicating the cast;
(e) leaving the cast to de-aerate;
(f) pouring the cast onto a surface and spreading the cast out to the desired thickness;
(g) drying the cast layer at a temperature of from −10 to 30° C. and a pressure of from 0.5 to 1 atm, until the residual water content of the film is from 0 to 20% by weight and a solid film is formed; and
(h) optionally, cutting the solid film into pieces of the desired size, further optionally placing these pieces into pouches, preferably wherein the pouches are made from PET-lined aluminium, sealing the pouches and further optionally, labelling them.

### DETAILED DESCRIPTION OF THE INVENTION

The present invention is concerned with a film, suitable for administration to an oral cavity, which can be used for delivery of a carrier system, comprising a carrier, a pathogen entry protein and an active pharmaceutical agent or a pharmaceutically acceptable salt thereof, to a human patient. Such a film may also be referred to as an oral dissolvable film (ODF) and/or an oral transmucosal film (OTF). The film is typically an alginate film which is applied by the patient themselves or another person, e.g. a medical practitioner, a nurse, a carer, a social worker, a colleague of the patient or a family member of the patient, to the mucosa of the oral cavity. The film is bioadhesive and adheres to the surface of the oral cavity upon application. After application, the alginate film begins to dissolve, releasing the active pharmaceutical ingredient. The present invention is particularly useful in the treatment of infectious disease.

For the avoidance of doubt, all alternative and preferred features relating to the film per se apply equally to the use of said film in the treatment of a human patient.

### Definitions

As defined herein, “room temperature” refers to a temperature of 25° C.

As defined herein, the term “oral cavity” is understood to mean the cavity of the mouth, and includes the inner upper and lower lips, all parts of the inner cheek, the sublingual area under the tongue, the tongue itself, as well as the upper and lower gums and the hard and soft palate.

As defined herein, the term “oral mucosa” is understood to mean the mucous membrane lining the inside of the mouth, and includes (but does not exclusively refer to) mucosa in the buccal, labial, sublingual, gingival or lip areas, the soft palate and the hard palate.

As defined herein, the term “ambient conditions” is understood to mean a temperature of 25° C., a pressure of 1 atm and in the presence of air of normal composition (i.e. 78% nitrogen, 21% oxygen, 0.93% argon and 0.04% carbon dioxide).

As defined herein, the term “carrier” refers to a composition capable of delivering a reagent to a desired compartment, e.g. a certain cell type, of the human body and is useful for providing and controlling release of drugs after administration. Carriers that are preferred in the context of the present invention are those that enclose a cavity. It is preferred that the API or pharmaceutically acceptable salt thereof is inside this cavity. Carriers may have a spherical or substantially spherical or non-spherical shape, and are preferably spherical or substantially spherical. To allow the desired uptake of the carrier system into the desired target area, e.g. a certain cell type, carriers typically have a diameter of less than 1000 μm, preferably less 500 μm, more preferably less than 200 μm, still more preferably less than 100 μm, yet more preferably less than 50 μm, even more preferably less than 20 μm, still more preferably less than 10 μm, further preferably less than 5 μm, yet further preferably less than 1 μm, even further preferably less than 500, still further preferably less than 200 nm and most preferably less than 100 nm. Said carrier can be used for systemic or local application. Preferred examples of such carriers are micro- or nanoparticles, e.g. liposomes, nanofibers, nanotubes, nanocubes, virosomes, or erythrocytes etc. The most preferred carrier is a liposome.

As used herein, the term “diameter” of a particle refers to the longest linear distance from one side of the particle to the opposite side of the particle, passing through the centre point of the particle. Carrier diameters may be measured by any suitable technique known to the skilled person, such as laser light diffraction and/or scanning electron microscopy.

As used herein, the term “invasin” refers to an intracellular membrane protein involved in bacterial adhesion of Enterobacteriaceae, preferably of the Yersinia, Edwardsiella, or Escherichia species, preferably Yersinia pseudotuberculosis, Yersinia pestis, Yersinia ruckeri, Yersinia enterocolitica. Yersinia rhodei, Yersinia similis, Escherichia coli (E. coli). Such bacterial adhesion proteins are characterized as “Invasins”, if they comprise an invasin consensus spanning amino acids 191 to 289 of SEQ ID NO: 2 or a sequence that shares at least 70%, more preferably at least 80%, and even more preferably at least 90% amino acid sequence identity to the consensus sequence over the entire length of the consensus sequence. A particularly preferred invasin is invasin A encoded by the inv gene of Yersinia pseudotuberculosis (see e.g. Gene Bank Accession No. M17448). This protein consists of 986 amino acid residues, and can be divided into two parts: the first region, consisting of the N-terminal region (or N-terminus), is located within the outer membrane of the bacterium, while the second part of the protein towards the C-terminal region (or C-terminus) is located extracellularly. The extracellular region of the protein has been shown to be the interaction site with β₁-integrin receptors of the host. As mentioned above, invasin is known to promote the attachment and uptake of Yersinia by microfold cells of the epithelial lining of the GI tract. Upon binding of invasin to β₁ integrin receptors on epithelial cells, a chain of signalling cascades provokes rearrangement of the cytoskeletal system that leads to protrusions of the host membrane which surround the bacterium, eventually internalizing it.

As used herein, the term “internalin” refers to a surface protein of Listeria monocytogenes. There exist two different internalins, InlA and InlB, encoded by two genes. InlA and InlB have common structural features, i.e. two repeat regions: the leucine-rich repeat regions and the B-repeat region, separated by a highly conserved inter-repeat region. The carboxy-terminal region of InlA contains an LPXTG motif, a signature sequence necessary for anchoring internalin on the bacterial surface and that internalin exposed on the surface is capable of promoting entry. InlB contains repeated sequences beginning with the amino acids GW, necessary to anchor InlB to the bacterial surface. Internalins are used by the bacteria to invade mammalian cells via cadherins or other transmembrane proteins of the host. InlA is necessary to promote Listeria entry into human epithelial cells, i.e. Caco-2 cells, wherein InlB is necessary to promote Listeria internalization in several other cell types, including hepatocytes, fibroblasts and epithelioid cells, such as Vero, HeLa, CHO, or HEp-2 cells.

As used herein, the term “mammalian target cell” refers to any cell which originates from a mammal. Further, the mammalian target cell can be in an infected condition wherein this infected condition is triggered by a pathogen invaded in said mammalian cell. Pathogens or infective agents are microorganisms, such as a virus, bacterium, prion, fungus or protozoan that causes disease in its host. A mammalian target cell is any cell from mammalian tissue which can be targeted by the carrier system disclosed herein.

As used herein, the term “liposomes” refers to spherical soft-matter vesicles consisting of one or more bilayers of amphiphilic molecules encapsulating a volume of aqueous medium. Preferred amphiphilic molecules are natural or synthetic lipids, phospholipids or mixtures thereof. The phospholipids may further contain cholesterol as mentioned in more detail below. Lipids used for the formation of liposomes of the invention consist of a hydrophilic head-group and hydrophobic tail; in excess in aqueous solutions, such lipids orient themselves so that hydrophilic head-groups are exposed to the aqueous phase while the hydrophobic hydrocarbon moieties (fatty acid chains having 10-24 carbon atoms and 0-6 double bonds in each chain) are forced to face each other within the bilayer. Therefore, the liposomes are able to entrap both hydrophilic and lipophilic/hydrophobic drugs-water-soluble drugs may be located in their internal or inter-bilayer aqueous spaces, while lipophilic/hydrophobic drugs may incorporate within the membrane itself. Cholesterol and/or its derivatives are quite often incorporated into the phospholipid membrane. These compounds arrange themselves within liposomes with hydroxyl groups oriented towards the aqueous surfaces and aliphatic chains aligned parallel to the acyl chains in the centre of the bilayer. The presence of cholesterol or derivatives thereof makes the membrane less ordered and slightly more permeable below the transition temperature of phospholipids, while above the transition temperature membranes containing cholesterol exhibit a more rigid/less fluid structure. On the basis of their structural properties, liposomes can vary widely in size which is an important parameter for circulation half-life. They may also vary in the number and position of lamellae present. Both liposome size and number of bilayers affect the degree of drug encapsulation in liposomes. According to the number of bilayers, liposomes can be divided into different categories. Unilamellar vesicles are structures in which the vesicle has a single phospholipid bilayer enclosing the aqueous core, and can be further divided into three important groups: small unilamellar vesicles (SUV) which have a size range of from 0.02 μm to 0.1 μm in diameter; large unilamellar vesicles (LUV) which have a size range of from 0.1 μm to 1 μm in diameter; and giant unilamellar vesicles, which have a size of more than 1 μm in diameter. Multilamellar vesicles (MLV) which usually consist of a population of vesicles covering a wide range of sizes more than 0.5 μm in diameter, each vesicle generally consisting of three or more concentric lamellae. Vesicles composed of just a few concentric lamellae are called oligolamellar vesicles (OLV). These vesicles are considered to be two bilayers, and range in size from 0.1 μm to 1 μm in diameter. Multivesicular vesicles (MVV) can also occur, wherein two or more vesicles are enclosed together in a nonconcentric manner within another larger one with a size range more than 0.1 μm in diameter. Liposomes can be classified according to their chemical characteristics. As mentioned, liposomes are composed of natural and or synthetic lipids, and may also contain other constituents such as cholesterol and hydrophilic polymer-conjugated lipids. The physicochemical characteristics of lipids composing the liposomal membrane, such as their fluidity, permeability and charge density, determine the behaviour of liposomes following their application or administration. The importance of liposome composition in their action as drug delivery systems has led to a composition-based classification system for liposomes. Conventional liposomes consist of neutral or negatively charged phospholipids and cholesterol, containing a hydrophilic drug encapsulated inside the liposome or hydrophobic drug incorporated into the liposome bilayer. Long-circulating liposomes (LCL) are liposomes functionalized with a protective polymer such as polyethyleneglycol (PEG) to avoid opsonization. Long-circulating immuno-liposomes are liposomes functionalized with both a protective polymer and antibody, which can be grafted to the liposome bilayer or attached to the distal end of the coupled polymer. Smart liposomes comprise liposomes with single or multiple modifications, such as attachment of a diagnostic label, incorporation of stimuli-sensitive lipids, incorporation of positively charged lipids which allow the functionalization with DNA, attachment of cell-uptake peptides, attachment of stimuli-sensitive polymer, or incorporation of viral components. In addition, all these types of liposomes can be loaded with magnetic-targeting particles, or with diagnostic markers, e.g. fluorescence markers, or gold or silver particles for imaging using electron microscopy.

As used herein, the term “molecule on the surface of a mammalian target cell” refers to any protein capable of specifically interacting with the pathogen entry protein. This term thus encompasses receptor molecules, i.e. a protein molecule which is usually found inside or on the surface of a cell that receives chemical signals from outside the cell. When such chemical signals bind to a receptor, they cause some form of cellular/tissue response, e.g. change in the electrical activity of the cell. In this sense, a receptor is a molecule that recognizes and responds to endogenous chemical signals, e.g. the acetylcholine receptor recognizes and responds to its endogenous ligand, acetylcholine. However sometimes in pharmacology, the term is also used to include other proteins that are drug targets, such as enzymes, transporters and ion channels. Receptor proteins are embedded in either the cell's plasma membrane (cell surface receptors), cytoplasm (cytoplasmic receptors), or in the nucleus (nuclear receptors). A molecule that binds to a receptor is called a ligand, and can be a peptide (short protein) or another small molecule such as a neurotransmitter, hormone, pharmaceutical drug, or toxin. The endogenously designated molecule for a particular receptor is referred to as its endogenous ligand. Each receptor is linked to a specific cellular biochemical pathway. While numerous receptors are found in most cells, each receptor will only bind to ligands of a particular structure, much like how locks will only accept specifically shaped keys. When a ligand binds to its corresponding receptor, it activates or inhibits the receptor's associated biochemical pathway. The structures of receptors are very diverse and can broadly be classified into the ionotropic receptors, G-protein-coupled receptors, kinase-linked and related receptors and nuclear receptors.

As used herein, the term “bacterium sequestering in a non-phagocytic cell” refers to a bacterium which has invaded the intracellular space of a host cell and exists therein in an abandoned part, i.e. a vacuole or capsule, typically to evade immune response, wherein the host cell is a non-phagocytic cell. Non-phagocytic cells comprise all type of cells which does not ingest and destroy foreign particles, bacteria, and cell debris.

As used herein, the term “pathogen” typically refers to an infectious agent (colloquially known as a germ). Pathogens thus include microorganisms such as viruses, bacteria, prions, fungi or protozoa, which cause disease in its host. The host may be an animal, a plant or a fungus.

As used herein, the term “Gram-negative bacteria” refers to a class of bacteria that do not retain the crystal violet stain used (contrarily to “Gram-positive bacteria”) in the Gram staining method of bacterial differentiation making positive identification possible. The thin peptidoglycan layer of their cell wall is sandwiched between an inner cell membrane and a bacterial outer membrane. In Gram staining, the outer lipid-based membrane of Gram-negative bacteria is removed by an alcohol solution which also decolorizes the then exposed peptidoglycan layer by dissolving away the previously applied crystal violet. A counterstain (safranin or fuchsine) is then added which recolorizes the bacteria red or pink. Gram-positive bacteria include Streptococcus, Staphylococcus, Bacillus, Clostridium, Corynebacterium and Listeria. Common Gram-negative bacteria include the proteobacteria, a major group of Gram-negative bacteria, including E. coli, Salmonella, Shigella, and other Enterobacteriaceae (Yersinia), Pseudomonas, Moraxella, Helicobacter, Stenotrophomonas, Bdellovibrio, acetic acid bacteria, and Legionella. A well-known Gram-negative bacterium is Yersinia pseudotuberculosis which is a facultative anaerobic, coccoid bacillus of the genus Yersinia from the Enterobacteriaceae family. It is motile at room temperature but non-motile at 37° C. The genome of Yersinia pseudotuberculosis contains one circular chromosome and two plasmids; one of the plasmids is responsible for the virulence of the bacteria, and the other one encodes mobilization information. Once it has achieved entry into Microfold cells (M-cells), epithelial cells or phagocytes, Yersinia pseudotuberculosis is enclosed in an acidic compartment called a Bacteria-containing vacuole (BCV). Y. pseudotuberculosis alters the endocytic pathway of this vacuole in order to avoid being destroyed, and replicates. Yersinia species, including Yersinia pseudotuberculosis and Yersinia enterocolitica, cause several GI disorders such as enteritis, colitis, diarrhea, lymphadenitis, and other associated disorders such as erythema nodosum, uveitis and septicemia. These bacteria promote their own uptake through the epithelial lining of the GI tract by interaction with M-cells, via a small bacterial membrane-bound protein called invasin. In this way, they gain access to the host lymphatic system by macrophages and cause inflammation of these tissues. Typical symptoms of systemic Yersinia pseudotuberculosis infection include joint or back pain, abdominal cramps and diarrhoea. Infection, in both local and systemic cases, can be treated by tetracyclines, aminoglycosides, chloramphenicol and third generation cephalosporins. Another Gram-negative species is Salmonella, a rod-shaped, predominantly motile enteric bacterium. The genome of Salmonella enterica contains one chromosome and plasmid. Salmonella enterica has an outer membrane consisting largely of lipopolysaccharides which protect the bacteria from the environment. Salmonella species are facultative intracellular pathogens that enter cells by manipulating the host's cytoskeletal elements and membrane trafficking pathways, which initiates an actin-mediated endocytic process called macropinocytosis via Salmonella-Invasion-Proteins (Sips). Intracellular bacteria replicate within a membrane-bound vacuole known as the Salmonella-containing vacuole. However, this bacterium can also replicate efficiently in the cytosol of epithelial cells; intracellular growth is therefore a product of both vacuolar and cytosolic replication. Salmonella enterica causes gastroenteritis in humans and other mammals. The disease is characterized by diarrhoea, abdominal cramps, vomiting and nausea, and generally lasts up to 7 days. Infections caused by Salmonella species are usually treated with aminoglycosides and chloramphenicol. Other Gram-negative bacteria include the proteobacteria, such as E. coli, Salmonella, Shigella and other Enterobacteriaceae, Pseudomonas, Moraxella, Helicobacter, Stenotrophomonas, Bdellovibrio, acetic acid bacteria, and Legionella. Other notable groups of Gram-negative bacteria include the cyanobacteria, spirochaetes, green sulfur, and green non-sulfur bacteria. Medically relevant Gram-negative cocci include the three organisms that cause a sexually transmitted disease (Neisseria gonorrhoeae), a meningitis (Neisseria meningitidis), and respiratory symptoms (Moraxella catarrhalis). Medically relevant Gram-negative bacteria include a multitude of species. Some of them cause primarily respiratory problems (Hemophilus influenzae, Klebsiella pneumoniae, Legionella pneumophila, Pseudomonas aeruginosa), primarily urinary problems (E. coli, Proteus mirabilis, Enterobacter cloacae, Serratia marcescens), or primarily gastrointestinal problems (Helicobacter pylori, Salmonella enteritidis, Salmonella typhi, Campylobacter jejuni). Gram-negative bacteria associated with hospital-acquired infections include Acinetobacter baumannii, which cause bacteremia, secondary meningitis, and ventilator-associated pneumonia in hospital intensive-care units.

As used herein, the term “covalently linked” describes two molecules connected by a covalent bond which is a chemical bond that involves the sharing of electron pairs and atoms. Commonly in protein/peptide chemistry, the N-terminus of a protein/peptide may be covalently linked to a carboxyl group of the linkage partner. Typically, the carboxylic groups of the cross-linking partner require activation prior to covalent bond formation using suitable reagents. To enhance the electrophilicity of the carboxylate group, the carboxylate group is chemically modified to transform one of the oxygen atoms into a superior leaving group. Several reagents are useful for this purpose, including N,N′-diisopropylcarbodiimide (DIC), N,N′-dicyclohexylcarbodiimide (DCC), N-(3-dimethylaminopropyl)-N-ethylcarbodiimide hydrochloride (EDC), sulfosuccinimide, N-hydroxybenzotriazole, N-hydroxysuccinimide (NETS), 4-(4,6-Dimethoxy-1,3,5-triazin-2-yl)-4-methylmorpholiniumchloride (DMTMM), maleidesters, glutaraldehyde, benzotriazol-1-yloxytris(dimethylamino)phosphonium hexafluorophosphate (BOP), 1-Cyano-2-ethoxy-2-oxoethylidenaminooxy)dimethylamino-morpholino-carbenium hexafluorophosphate (COMU), 3-(diethoxyphosphoryloxy)-1,2,3-benzotriazin-4(3H)-one (DEPBT), 1-[Bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphate (HBTU), O-(1H-6-Chlorobenzotriazole-1-yl)-1,1,3,3-tetramethyluronium hexafluorophosphate (HCTU), benzotriazol-1-yl-oxytripyrrolidinophosphonium hexafluorophosphate (PyBOP), (7-Azabenzotriazol-1-yloxy)tripyrrolidinophosphonium hexafluorophosphate (PyAOP), (Ethyl cyano(hydroxyimino)acetato-O²)tri-1-pyrrolidinylphosphonium hexafluorophosphate (PyOxim) or O—(N-Succinimidyl)-1,1,3,3-tetramethyluronium tetrafluoroborate (TSTU). The reaction between the N-terminus of the protein/peptide and the carboxylate group results in the formation of an amide.

As used herein, the term “biologically active moiety” refers to any moiety that is derived from a biologically active molecule by abstraction of a hydrogen radical. A “biologically active molecule” is any molecule capable of inducing a biochemical response when administered in vivo. Typically, the biologically active molecule is capable of producing a local or systemic biochemical response when administered to an animal (or, preferably, a human); preferably the local or systemic response is a therapeutic activity. Preferred examples of biologically active molecules include drugs, peptides, proteins, peptide mimetics, antibodies, antigens, DNA, RNA, mRNA, small interfering RNA, small hairpin RNA, microRNA, PNA, foldamers, carbohydrates, carbohydrate derivatives, non-Lipinski molecules, synthetic peptides and synthetic oligonucleotides, and most preferably small molecule drugs.

As used herein, the term “small molecule drug” refers to a chemical compound which has known biological effect on an animal, such as a human. Typically, drugs are chemical compounds which are used to treat, prevent or diagnose a disease. Preferred small molecule drugs are biologically active in that they produce a local or systemic effect in animals, preferably mammals, more preferably humans. The small molecule drug may be referred to as a “drug molecule” or “drug”. Typically, the drug molecule has Mw less than or equal to about 5 kDa. Preferably, the drug molecule has Mw less than or equal to about 1.5 kDa. A more complete, although not exhaustive, listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, edited by Susan Budavari et al., CRC Press, 1996, both of which are incorporated herein by reference in their entirety.

As used herein, the term “peptides” refers to biologically occurring or synthetic short chains of amino acid monomers linked by peptide (amide) bonds. The covalent chemical bonds are formed when the carboxyl group of one amino acid reacts with the amino group of another. The shortest peptides are dipeptides, consisting of 2 amino acids joined by a single peptide bond, followed by tripeptides, tetrapeptides, etc. A polypeptide is a long, continuous, and unbranched peptide chain. Hence, peptides fall under the broad chemical classes of biological oligomers and polymers, alongside nucleic acids, oligosaccharides and polysaccharides, etc.

As used herein, the term “amino acid” refers to any natural or synthetic amino acid, that is, an organic compound comprising carbon, hydrogen, oxygen and nitrogen atoms, and comprising both amino (—NH₂) and carboxylic acid (—COOH) functional groups. Typically, the amino acid is an α-, β-, γ- or δ-amino acid. Preferably, the amino acid is one of the twenty-two naturally occurring proteinogenic α-amino acids. Alternatively, the amino acid is a synthetic amino acid selected from α-Amino-n-butyric acid, Norvaline, Norleucine, Alloisoleucine, t-leucine, α-Amino-n-heptanoic acid, Pipecolic acid, α,β-diaminopropionic acid, α,γ-diaminobutyric acid, Ornithine, Allothreonine, Homocysteine, Homoserine, β-Alanine, 3-Amino-n-butyric acid, 3-Aminoisobutyric acid, γ-Aminobutyric acid, α-Aminoisobutyric acid, isovaline, Sarcosine, N-ethyl glycine, N-propyl glycine, N-isopropyl glycine, N-methyl alanine, N-ethyl alanine, N-methyl β-alanine, N-ethyl β-alanine, isoserine, α-hydroxy-γ-aminobutyric acid, Homonorleucine, O-methyl-homoserine, O-ethyl-homoserine, selenohomocysteine, selenomethionine, selenoethionine, Carboxyglutamic acid, Hydroxyproline, Hypusine, Pyroglutamic acid, aminoisobutyric acid, dehydroalanine, β-alanine, γ-Aminobutyric acid, δ-Aminolevulinic acid, 4-Aminobenzoic acid, citrulline, 2,3-diaminopropanoic acid and 3-aminopropanoic acid. An amino acid which possess a stereogenic centre may be present as a single enantiomer or as a mixture of enantiomers (e.g. a racemic mixture). Preferably, if the amino acid is an α-amino acid, the amino acid has L stereochemistry about the α-carbon stereogenic centre.

As used herein, the term “proteins” refers to biological molecules comprising polymers of amino acid monomers which are distinguished from peptides on the basis of size, and as an arbitrary benchmark can be understood to contain approximately 50 or more amino acids. bound to ligands such as coenzymes and cofactors, or to another protein or other macromolecule (DNA, RNA, etc.), or to complex macromolecular assemblies. Proteins perform a vast array of functions within living organisms, including catalyzing metabolic reactions, replicating DNA, responding to stimuli, and transporting molecules from one location to another. Proteins differ from one another primarily in their sequence of amino acids, which is dictated by the nucleotide sequence of their genes, and which usually results in folding of the protein into a specific three-dimensional structure that determines its activity.

As used herein, the term “peptide mimetics” refers to small protein-like chains designed to mimic a peptide. They typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. This can have a role in the development of drug-like compounds from existing peptides. These modifications involve changes to the peptide that will not occur naturally (such as altered backbones and the incorporation of non-natural amino acids).

As used herein, the term “nucleic acid” refers to polymeric or oligomeric macromolecules, or large biological molecules, essential for all known forms of life. Nucleic acids include DNA, RNA (e.g. mRNA, siRNA, shRNA, miRNA and piRNA), PNA and other synthetic nucleic acids such as morpholino and locked nucleic acid (LNA), glycol nucleic acid (GNA) and threose nucleic acid (TNA).

As used herein, the term “mRNA” refers to messenger RNA, a family of RNA molecules that convey genetic information from DNA to the ribosome, where they specify the amino acid sequence of the protein products of gene expression. Following transcription of primary transcript mRNA (known as pre-mRNA) by RNA polymerase, processed, mature mRNA is translated into a polymer of amino acids: a protein. As in DNA, mRNA genetic information is in the sequence of nucleotides, which are arranged into codons consisting of three bases each. Each codon encodes for a specific amino acid, except the stop codons, which terminate protein synthesis. This process of translation of codons into amino acids requires two other types of RNA: transfer RNA (tRNA), that mediates recognition of the codon and provides the corresponding amino acid, and ribosomal RNA (rRNA), that is the central component of the ribosome's protein-manufacturing machinery.

As used herein, the term “small interfering RNA” (siRNA) refers to a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation. siRNA also acts in RNAi-related pathways, e.g. as an antiviral mechanism or in shaping the chromatin structure of a genome.

As used herein, the term “small hairpin RNA” (shRNA) refers to an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

As used herein, the term “micro RNA” (miRNA) refers to a small non-coding RNA molecule (containing about 22 nucleotides) found in plants, animals, and some viruses, which functions in RNA silencing and post-transcriptional regulation of gene expression.

As used herein, the term “piRNA” refers to short RNAs that typically comprise 26-31 nucleotides and derive their name from so-called piwi proteins they bind to.

As used herein, the term “PNA” refers to peptide nucleic acid, an artificially synthesized polymer similar to DNA or RNA invented by Peter E. Nielsen (Univ. Copenhagen), Michael Egholm (Univ. Copenhagen), Rolf H. Berg (Risø National Lab), and Ole Buchardt (Univ. Copenhagen) in 1991. PNA's backbone is composed of repeating N-(2-aminoethyl)-glycine units linked by peptide bonds. The various purine and pyrimidine bases are linked to the backbone by a methylene bridge (—CH₂—) and a carbonyl group (—(C═O)—).

As used herein, the term “DNA” refers to deoxyribonucleic acid and derivatives thereof, the molecule that carries most of the genetic instructions used in the development, functioning and reproduction of all known living organisms and many viruses. Most DNA molecules consist of two biopolymer strands coiled around each other to form a double helix. The two DNA strands are known as polynucleotides since they are composed of simpler units called nucleotides. Each nucleotide is composed of a nitrogen-containing nucleobase—cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T, and C with G), hydrogen bonds bind the nitrogenous bases of the two separate polynucleotide strands to make double-stranded DNA.

As used herein, the term “foldamer” refers to a discrete chain molecule or oligomer that folds into a conformationally ordered state in solution. They are artificial molecules that mimic the ability of proteins, nucleic acids, and polysaccharides to fold into well-defined conformations, such as helices and β-sheets. The structure of a foldamer is stabilized by non-covalent interactions between nonadjacent monomers.

As used herein, the term “carbohydrate” refers to biological molecule consisting of carbon (C), hydrogen (H) and oxygen (O) atoms, usually with a hydrogen:oxygen atom ratio of 2:1 (as in water); in other words, with the empirical formula Cₘ(H₂O)ₙ (where m could be different from n). Some exceptions exist; for example, deoxyribose, a sugar component of DNA, has the empirical formula C₅H₁₀O₄. Carbohydrates are technically hydrates of carbon; structurally it is more accurate to view them as polyhydroxy aldehydes and ketones. The term is most common in biochemistry, where it is a synonym of saccharide, a group that includes sugars, starch, and cellulose. The saccharides are divided into four chemical groups: monosaccharides, disaccharides, oligosaccharides, and polysaccharides.

As used herein, the term “non-Lipinski molecules” refers to molecules that do not conform to Lipinski's rule of five (also known as the Pfizer's rule of five or simply the Rule of five (R05)), which is a rule of thumb to evaluate drug-likeness or to determine whether a chemical compound with a certain pharmacological or biological activity has properties that would make it a likely orally active drug in humans. The rule was formulated by Christopher A. Lipinski in 1997, based on the observation that most orally administered drugs are relatively small and moderately lipophilic molecules. The rule describes molecular properties important for a drug's pharmacokinetics in the human body, including their absorption, distribution, metabolism, and excretion (“ADME”). However, the rule does not predict if a compound is pharmacologically active.

As used herein, the term “release kinetic” refers to the release of the API or pharmaceutically acceptable salt thereof from the carrier system or the carrier from the pharmaceutical composition of the present invention to its molecular target. Pharmacokinetics comprises the determination of the fate of a substance administered to a living organism and may comprise different kinetics, i.e. rapid release, prolonged or delayed release or sustained release.

As used herein, the term “pathogen entry protein” refers to a protein which facilitates entry of pathogenic organisms, preferably a bacterium, into a particular host cell and facilitates infection of said cell. Fragments of such proteins, i.e. proteins carrying N-terminal, C-terminal, and/or internal deletions, may still be capable of mediating entry into a particular host cell. Successful establishment of intracellular infection by bacterial pathogens requires first an adhesion to the host cells and then cellular invasion, frequently followed by intracellular multiplication, dissemination to the other tissues, or persistence. Bacteria used monomeric adhesins/invasins or highly sophisticated macromolecular machines such as type III secretion system to establish a complex host/pathogen interaction which leads to subversion of cellular functions and establishment of disease. Many pathogenic organisms, for example many bacteria, must first bind to host cell surfaces and several bacterial and host molecules that are involved in the adhesion of bacteria to host cells have been identified. Often, the host cell receptors for bacteria are essential proteins for other functions. Due to the presence of a mucous lining and of anti-microbial substances around some host cells, it is difficult for certain pathogens to establish direct contact-adhesion. Some virulent bacteria produce proteins that either disrupt host cell membranes or stimulate their own endocytosis or macro-pinocytosis into host cells. These virulence factors allow the bacteria to enter host cells and facilitate entry into the body across epithelial tissue layers at the body surface. One purpose of the carrier system utilised in the present invention is to deliver active agents, e.g. hydrophilic antipathogenic agents like antibiotics or cytostatics, loaded onto or into the carrier and using a pathogen entry protein and its invasion mechanism accessing a mammalian target cell which is in an infected state.

As used herein, the term “antibiotic” refers to an agent that is capable of killing or at least inhibiting growth of microrganisms, preferably of bacteria. Antibiotics can be selected from: β-lactam antibiotics, e.g. penicillins comprising benzylpenicillin, phenoxymethylpenicillin, piperacillin, mezlocillin, ampicillin, amoxicillin, flucloxacillin, methicillin, oxacillin; β-lactamase inhibitors e.g. clavulanic acid, sulbactam, tazobactam, sultamicillin; monobactams, e.g. aztreonam; cephalosporins, e.g. cefazolin, cefalexin, loracarbef, cefuroxime, cefotiam, cefaclor, cefotaxime, ceftriaxone, cefepime, ceftazidime, cefixime, cefpodoxime, ceftibuten; carbapenems, e.g. imipenem, meropenem, ertapenem; lipopeptides, e.g. daptomycin; glycopeptides, e.g. bleomycin, vancomycin, teicoplanin; aminoglycosides, e.g. gentamicin, dibekacin, sisomicin, tobramycin, amikacin, kanamycin, neomycin, streptomycin, netilmicin, apramycin, paromomycin, spectinomycin, geneticin; oxazolidinediones, e.g. linezolid; glycylcyclines, e.g. tigecycline; polypeptides, e.g. polymyxin; polyketides, e.g. tetracyclines comprising tetracycline, oxytetracycline, minocycline, doxycycline, chlortetracycline, rolitetracycline or macrolides comprising erythromycin, azithromycin, clarithromycin, roxythromycin; ketolides, e.g. telithromycin; quinolones, e.g. ciprofloxacin, norfloxacin, ofloxacin; moxifloxacin, enoxacin, gatifloxacin, sparfloxacin, pefloxacin, fleroxacin, levofloxacin, trovafloxacin; sulphonamides, e.g. sulfamethoxazole, sulfacarbamide, sulfacetamide, sulfamethylthiazole, sulfadiazine, sulfamethoxozole, sulfasalazine; or a pharmaceutically acceptable salt of any of the foregoing.

As used herein, the term “cytostatic” refers to chemical substances, especially one or more anti-cancer drugs or so-called chemotherapeutic agents. It is noted that some antibiotics, e.g. sulfadicramide, or sulfadimethoxine, also have cytostatic activity and are thus also included in the list of preferred cytostatics. Cytostatics include alkylating agents, anti-metabolites, anti-microtubule agents, topoisomerase inhibitors, cytotoxic antibiotics, anti-metabolites, epothilones, nuclear receptor agonists and antagonists, anti-androgens, anti-estrogens, platinum compounds, hormones and antihormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneamines, methylmelamines, trazines, vinca alkaloids, pyrimidine analogs, purine analogs, alkylsulfonates, folic acid analogs, anthracenediones, substituted urea, methylhydrazine derivatives. Preferred cytostatics include acediasulfone, aclarubicin, ambazone, aminoglutethimide, L-asparaginase, azathioprine, bleomycin, busulfan, calcium folinate, carboplatin, carpecitabine, carmustine, celecoxib, chlorambucil, cis-platin, cladribine, cyclophosphamide, cytarabine, dacarbazine, dactinomycin, dapsone, daunorubicin, dibrompropamidine, diethylstilbestrol, docetaxel, doxorubicin, enediynes, epirubicin, epothilone B, epothilone D, estramustin phosphate, estrogen, ethinylestradiol, etoposide, flavopiridol, floxuridine, fludarabine, fluorouracil, fluoxymesterone, flutamide, fosfestrol, furazolidone, gemcitabine, gonadotropin releasing hormone analog, hexamethylmelamine, hydroxycarbamide, hydroxymethylnitrofurantoin, hydroxyprogesteronecaproate, hydroxyurea, idarubicin, idoxuridine, ifosfamide, interferon a, mnotecan, leuprolide, lomustine, lurtotecan, mafenide sulfate, olamide, mechlorethamine, medroxyprogesterone acetate, megastrol acetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, bleomycin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, acriflavinium chloride, semustine, streptozotocin, sulfacarbamide, sulfacetamide, sulfachloropyridazine, sulfadiazine, sulfadicramide, sulfadimethoxine, sulfaethidole, sulfafurazole, sulfaguanidine, sulfaguanole, sulfamethizole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosterone propionate, thioguanine, thiotepa, tinidazole, topotecan, triaziquone, treosulfan, trimethoprim, trofosfamide, UCN-01, vinblastine, vincristine, vindesine, vinblastine, vinorelbine, zorubicin, or a pharmaceutically acceptable salt of any of the foregoing.

As defined herein, the term “API” refers to the form of the API in which the molecules are present in neutral (i.e. unionized) form. The term “pharmaceutically acceptable salt of the API” refers to any salt of the API compound.

### Films of the Present Invention

The present invention provides films suitable for administration to an oral cavity comprising:
(i) an alginate salt of a monovalent cation or a mixture of alginate salts containing at least one alginate salt of a monovalent cation; and
(ii) a carrier system comprising:
   (a) a carrier,
   (b) a pathogen entry protein or fragment thereof, which specifically binds to a molecule on the surface of a mammalian target cell of said pathogen and which is covalently linked to the surface of said carrier, and
   (c) at least one active pharmaceutical ingredient (API) or pharmaceutically acceptable salt thereof.

The function of said alginate salt of a monovalent cation or mixture of alginate salts containing at least one alginate salt of a monovalent cation within the film is to act as a film-forming agent. As used herein, the term “film-forming agent” refers to a chemical or group of chemicals that form a pliable, cohesive and continuous covering when applied to a surface.

Alginate, the salt of alginic acid, is a linear polysaccharide naturally produced by brown seaweeds (Phaeophyceae, mainly Laminaria). Typically the alginate employed in the present invention comprises from 100 to 3000 monomer residues linked together in a flexible chain. These residues are of two types, namely β-(1,4)-linked D-mannuronic acid (M) residues and α-(1,4)-linked L-guluronic acid (G) residues. Typically, at physiological pH, the carboxylic acid group of each residue in the polymer is ionised. The two residue types are epimers of one another, differing only in their stereochemistry at the C5 position, with D-mannuronic acid residues being enzymatically converted to L-guluronic acid residues after polymerization. However, in the polymer chain the two residue types give rise to very different conformations: any two adjacent D-mannuronic acid residues are ⁴C₁-diequatorially linked whilst any two adjacent L-guluronic acid residues are ⁴C₁-diaxially linked, as illustrated in Formula (I) below.

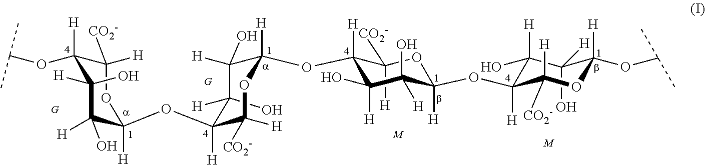

Typically in the alginate polymer, the residues are organised in blocks of identical or strictly alternating residues, e.g. MMMMM . . . , GGGGG . . . or GMGMGM . . . . Different monovalent and polyvalent cations may be present as counter ions to the negatively-charged carboxylate groups of the D-mannuronic acid and L-guluronic acid residues of the alginate polymer. Typically, the film comprises an alginate salt wherein the counter ions of the alginate polymer are monovalent cations. The cations which are the counterions of a single alginate polymer molecule may all be the same as one another or may be different to one another. Preferably, the counterions of the alginate polymer are selected from Na⁺, K⁺ and NH₄⁺. More preferably, the counterions of the alginate polymer are Na^{t}. Alternatively, the film may comprise a mixture of alginate salts containing at least one alginate salt of a monovalent cation. The mixture of alginate salts may comprise an alginate salt of a cation selected from Na⁺, K⁺ and NH₄⁺. Thus, typically, the alginate chains are not cross-linked, i.e. there is no, or substantially no, ionic cross-linking between the alginate strands. Ionic cross-linking of alginates results from the presence of divalent counterions. “Substantially no” cross-linking can be taken to mean that fewer than 10% by weight of the alginate polymer chains in the film are cross-linked, preferably fewer than 5% by weight, more preferably fewer than 2% by weight, still more preferably fewer than 1% by weight, yet more preferably fewer than 0.5% by weight, and most preferably fewer than 0.1% by weight. Thus, preferably, the films of the present invention comprise no alginate salts of a divalent cation.

Typically, the film comprises an alginate composition which has a dynamic viscosity, as measured on a 10% aqueous solution (w/w) thereof at a temperature of 20° C. with a Brookfield LVF viscometer (obtained from Brookfield Engineering Laboratories, Inc.), using a spindle No. 2 at a shear rate of 20 rpm, of 100-1000 mPa·s, or 200-800 mPa·s, or 300-700 mPa·s.

Preferably, the film comprises an alginate composition having a mean guluronate (G) content of from 50 to 85%, more preferably from 60 to 80%, and most preferably from 65 to 75% by weight. Preferably, the film comprises an alginate composition having a mean maluronate (M) content of from 15 to 50%, more preferably from 20 to 40%, and most preferably from 25 to 35% by weight. Preferably, the film comprises an alginate composition having a weight average molecular weight ranging from 20,000 g/mol to 90,000 g/mol, such as from 30,000 g/mol to 90,000 g/mol, or from 35,000 g/mol to 85,000 g/mol, or from 40,000 g/mol to 70,000 g/mol, or from 40,000 g/mol to 50,000 g/mol. Typically, the film comprises an alginate composition having a mean guluronate (G) content of from 50 to 85%, a mean maluronate (M) content of from 15 to 50%, and a weight average molecular weight ranging from 20,000 g/mol to 90,000 g/mol. Preferably, the film comprises an alginate composition having a mean guluronate (G) content of from 50 to 85%, a mean maluronate (M) content of from 15 to 50%, and a weight average molecular weight ranging from 30,000 g/mol to 90,000 g/mol. More preferably, the film comprises an alginate composition having a mean guluronate (G) content of from 60 to 80%, a mean maluronate (M) content of from 20 to 40%, and a weight average molecular weight ranging from 30,000 g/mol to 90,000 g/mol. Most preferably, the film comprises an alginate composition having a mean guluronate (G) content of from 65 to 75%, a mean maluronate (M) content of from 25 to 35%, and a weight average molecular weight ranging from 30,000 g/mol to 90,000 g/mol. Without wishing to be bound by any particular theory, it is believed that it is a combination of both (a) the particular mean relative proportions of maluronate and guluronate in the alginate composition and (b) the particular weight average molecular weight of the alginate composition that endow the film with its desirable bioadhesive properties.

The alginate salt of a monovalent cation or the mixture of alginate salts containing at least one alginate salt of a monovalent cation may be the sole film-forming agent present in the film. Alternatively, the film may comprise one or more further film-forming agents in addition to the alginate salt of a monovalent cation or the mixture of alginate salts containing at least one alginate salt of a monovalent cation.

It is preferred that the film comprises Protanal® LFR 5/60 or Protanal® LF 10/60 (both commercially available sodium alginate products from FMC BioPolymer) as the alginate salt. Protonal® LFR 5/60 is a low molecular weight and low viscosity sodium alginate extracted from the stem of Laminaria hyperborean. Protanal® LF 10/60 is a sodium alginate having a G/M % ratio of 65-75/25-35 and a viscosity of from 20-70 mPas as measured on a 1% aqueous solution thereof at a temperature of 20° C. with a Brookfield LVF viscometer, using a spindle No. 2 at a shear rate of 20 rpm. Protanal® LF 10/60 has both a higher weight average molecular weight and a higher viscosity than Protanal® LFR 5/60.

Without wishing to be bound by any particular theory, a film comprising a higher viscosity alginate salt is believed to have a longer residence time (i.e. dissolving time) after application to the oral cavity via adhesion to a mucous membrane of said cavity than a film comprising a lower viscosity alginate salt of a similar thickness. It is contemplated that the viscosity of the alginate composition within the film may be adjusted by mixing any number of alginates having different viscosities. Typically, a film of about 1 mm thickness comprising Protanal® LFR 5/60 as the sole alginate component has a residence time of approximately 3-10 minutes after adhesion to a mucous membrane of the oral cavity. In contrast, a film of about 1 mm thickness comprising Protanal® LF 10/60 as the sole alginate component has a residence time of approximately 30 minutes after adhesion to a mucous membrane of the oral cavity.

Therefore, if a long residence time of the film within the oral cavity is desired, it is generally preferred that the film comprises Protanal® LF 10/60 as the alginate salt. However, compared to films comprising Protanal® LFR 5/60 as the alginate salt, films comprising Protanal® LF 10/60 as the alginate salt typically exhibit inferior adhesion properties when applied to a mucous membrane of the oral cavity. More generally, it is believed that film-forming agents having longer average chain lengths exhibit poorer adhesion to mucosa than film-forming agents having shorter average chain lengths. Without wishing to be bound by any particular theory, it is believed that better mucoadhesion of a film to the mucous membrane of the oral cavity enables a more efficient delivery of any active ingredients contained within the film to their site of action. Therefore, if a long residence time of the film within the oral cavity is not particularly necessary, it may be preferable to use Protanal® LFR 5/60 as the alginate salt.

It is particularly preferred that the film comprises Protanal® LFR 5/60 as the alginate salt.

The film may also comprise a film-forming agent other than the alginate salt of a monovalent cation or the mixture of alginate salts containing at least one alginate salt of a monovalent cation. Such other film-forming agents include agents such as poly(vinyl pyrrolidone) (PVP), hydroxypropylmethylcellulose (HPMC), pullulan, and so forth. However, if any other film-forming agent is present in the film in addition to the alginate salt of a monovalent cation or the mixture of alginate salts containing at least one alginate salt of a monovalent cation, then typically the alginate salt of a monovalent cation or the mixture of alginate salts containing at least one alginate salt of a monovalent cation will be present in the film in excess over any other film-forming agent present. Preferably, the ratio (by weight) of the alginate salt of a monovalent cation or the mixture of alginate salts containing at least one alginate salt of a monovalent cation present in the film to the combined total of all other film-forming agents (such as PVP and/or pullulan) present in the film is 1:1 or greater, or 2:1 or greater, or 3:1 or greater, or 4:1 or greater, or 5:1 or greater, or 10:1 or greater, or 20:1 or greater, or 50:1 or greater, or 100:1 or greater, or 500:1 or greater, or 1000:1 or greater, or 10000:1 or greater. Preferably, the alginate salt of a monovalent cation or the mixture of alginate salts containing at least one alginate salt of a monovalent cation will constitute at least 50% by weight of the total of the film-forming agents present in the film, more preferably at least 60% by weight, at least 70% by weight, at least 80% by weight, at least 90% by weight, at least 95% by weight, at least 98% by weight, at least 99% by weight, at least 99.5% by weight, at least 99.9% by weight, at least 99.95% by weight, or at least 99.99% by weight of the total of the film-forming agents present in the film.

Preferably, the alginate salt of a monovalent cation or the mixture of alginate salts containing at least one alginate salt of a monovalent cation is substantially the only film-forming agent present in the film. More preferably, the alginate salt of a monovalent cation or the mixture of alginate salts containing at least one alginate salt of a monovalent cation is the only film-forming agent present in the film. Alternatively, the film does not comprise any, or substantially any, poly(vinyl pyrrolidone). Alternatively, the film does not comprise any, or substantially any, pullulan. Alternatively, the film does not comprise any, or substantially any, hydroxypropylmethylcellulose.

As used herein, a reference to a film that does not comprise “substantially any” of a specified component refers to a film that may contain trace amounts of the specified component, provided that the specified component does not materially affect the essential characteristics of the film. Typically, therefore, a film that does not comprise substantially any of a specified component contains less than 5 wt % of the specified component, preferably less than 1 wt % of the specified component, most preferably less than 0.1 wt % of the specified component.

It is a finding of the present invention that the use of an alginate salt of a monovalent cation or a mixture of alginate salts containing at least one alginate salt of a monovalent cation as the film-forming agent has benefits over the use of alternative film-forming agents, such as PVP, HPMC and/or pullulan. In particular, the use of alginate as the primary film-forming agent ensures that the films of the present invention have superior adhesive properties over films comprising primarily other film-forming agents such as PVP, HPMC or pullulan. The films of the present invention are bioadhesive; that is to say that the films of the present invention can firmly adhere to a moist surface (i.e. mucosa) in the oral cavity of a mammal subject before it has fully dissolved. Films in which alginate is not the primary film-forming agent do not generally have this desirable property. A further advantageous finding of the present invention is that the choice of alginate as the primary film-forming agent enables therapeutically effective doses of an active pharmaceutical ingredient (e.g., ketamine) to be loaded into the films whilst retaining homogeneity and other desirable physical properties of the films.

Typically, the film comprises from 15% to 99% by weight of the alginate salt of a monovalent cation or the mixture of alginate salts containing at least one alginate salt of a monovalent cation, preferably from 18% to 95% by weight, more preferably from 20% to 93% by weight, still more preferably from 25% to 90% by weight, and most preferably from 30% to 80% by weight.

The film according to the present invention may also contain a residual water content. Typically, the film comprises from 0% to 20% by weight of residual water. More typically, the film comprises from 5% to 15% by weight of residual water. Preferably, the film comprises from 9% to 11% by weight of residual water. Most preferably, the film comprises about 10% by weight of residual water. Typically, the low water content of the film distinguishes the film from pastes or gels (e.g. hydrogels), which typically have higher water contents. Thus, typically, the film of the present invention is not a paste. Typically, the film of the present invention is not a gel.

The film also comprises a carrier system comprising (a) a carrier, (b) a pathogen entry protein or fragment thereof, which specifically binds to a molecule on the surface of a mammalian target cell of said pathogen and which is covalently linked to the surface of said carrier, and (c) at least one active pharmaceutical ingredient (API) or pharmaceutically acceptable salt thereof.

Carrier systems suitable for use in the present invention include the systems described in WO 2016/024008, which is incorporated herein by reference in its entirety.

The pathogen entry protein is covalently linked, either directly or via a linker, to the surface of said carrier. The surface is preferably the outer surface of the carrier. The pathogen entry protein may be linked to any molecule on the surface of the carrier. Thus, the pathogen entry protein molecules may all be bound to the same type of molecule on the surface of the carrier. Alternatively, different pathogen entry protein molecules may be bound to different types of molecules on the surface of the carrier. A linker is a chemical spacer that increases the distance between the two entities linked. Typically a linker also improves the flexibility of motion between the two entities linked. The skilled person would be well aware of suitable linkers for attaching proteins to carriers such as liposomes or polymersomes. The linker group can be substantially any suitable multivalent organic group. It may be straight or branched. Merely by way of example, the linker group L may be an organic group having a molecular weight of 2000 or less, preferably 1500 or less, and more preferably 1000 or less. Preferred linkers include peptide linkers, which can be incorporated, e.g. at the N- or C-terminus of the pathogen entry protein. To provide improved flexibility, typically small amino acids are used in these peptide linkers, preferably selected from G, A, S, L, I, and V, and more preferably from G, A, and S.

The carrier system itself can provide different forms of release kinetics according to the physical and chemical properties of the carrier and the chemical interaction between the carrier and the API or pharmaceutically acceptable salt thereof. Depending on the carrier and type of chemical interaction the mode of release can be selected from rapid release, sustained release, or delayed release. The API or pharmaceutically acceptable salt thereof can be incorporated in the carrier system of the invention in different ways. It is preferred that it is incorporated in a way that leads to release once the carrier system reaches its target area, e.g. enters the target cell. To that end, the API or pharmaceutically acceptable salt thereof can be covalently or non-covalent linked to the carrier. If the link is covalent, it is preferred that the linkage is cleaved in the intracellular environment. If the API or pharmaceutically acceptable salt thereof is hydrophilic, it is preferred that it is incorporated within (i.e. is inside) a cavity of the carrier system. Alternatively, if the API or pharmaceutically acceptable salt thereof is hydrophobic, it is preferred that it is incorporated within a lipophilic membrane of the carrier, e.g. a phospholipid bilayer in a liposome.

Preferably, the carrier is selected from micro- or nanospheres, i.e. nanoparticles or liposomes, nanofibers, nanotubes, nanocubes, virosomes, or erythrocytes. Most preferably, the carrier is a liposome. The liposome may typically be a unilamellar or multilamellar liposome and/or neutral, positively or negatively charged liposomes.

Preferably, the carrier is covalently linked to the C-terminus, N-terminus or an amino acid side chain of the pathogen entry protein, more preferably via the N-terminus of the pathogen entry protein. As set out above, preferably the carrier is a liposome. In this case, the pathogen entry protein is typically covalently linked to one of the amphiphilic molecules comprised in the lipid layer(s) of the liposome. Preferably, the covalent link is between (i) the hydrophilic part of the amphiphilic molecule and (ii) the C-terminus, N-terminus or an amino acid side chain, more preferably the N-terminus, of the pathogen entry protein. This ensures that the pathogen entry protein is readily accessible on the surface of the carrier, e.g. the liposome. This is preferred to mediate the entry function of the pathogenic entry protein. Preferred examples of lipids for covalently connecting pathogenic entry proteins include 1,2-diaplmitoyl-sn-glycero-3-phosphocholine (DPPC), cholesterol, and 1,2-dihexadecanoyl-sn-glycero-3-phosphoethanolamine-N-(glutaryl) sodium salt.

The amphiphilic molecule, preferably the lipid that is covalently attached to the pathogen entry protein (also referred to as the “anchor molecule”) may be used solely to form the liposome or may be used in admixture with other amphiphilic molecules forming the liposome. Typically the anchor molecule constitutes less than 50 wt % of the total weight of the amphiphilic molecules (preferably lipids) forming the liposome, preferably less than 30 wt %, more preferably less than 20 wt %, yet more preferably less than 10 wt %, even more preferably less than 9 wt %, still more preferably less than 8 wt %, and most preferably less than 7 wt %.

In a particularly preferred aspect, the pathogen entry protein is covalently linked to a liposome comprising 1,2-diaplmitoyl-sn-glycero-3-phosphocholine (DPPC), cholesterol and 1,2-dihexadecanoyl-sn-glycero-3-phosphoethanolamine-N-(glutaryl) sodium salt. Preferably, the ratio of DPPC:cholesterol is from 1:20 to 20:1, more preferably from 1:10 to 10:1, yet more preferably from 1:5 to 5:1, still more preferably from 1:2 to 4:1, even more preferably from 1:1 to 3:1 and most preferably about 2:1. Preferably, the ratio of DPPC:1,2-dihexadecanoyl-sn-glycero-3-phosphoethanolamine-N-(glutaryl) sodium salt is from 1:10 to 100:1, more preferably from 1:5 to 50:1, yet more preferably from 1:2 to 30:1, still more preferably from 1:1 to 25:1, even more preferably from 1:5 to 20:1, and most preferably about 10:1. Preferably, the ratio of cholesterol:1,2-dihexadecanoyl-sn-glycero-3-phosphoethanolamine-N-(glutaryl) sodium salt is from 1:20 to 50:1, more preferably from 1:10 to 30:1, yet more preferably from 1:5 to 25:1, still more preferably from 1:2 to 20:1, even more preferably from 1:1 to 10:1, and most preferably about 5:1. Thus, most preferably DPPC, cholesterol and 1,2-dihexadecanoyl-sn-glycero-3-phosphoethanolamine-N-(glutaryl) sodium salt are present in a molar ratio of about 6:3:0.6. It is further preferred that the pathogen entry protein or fragment thereof is linked to the liposome either via its N-terminus, C-terminus or a side chain, more preferably via its N-terminus to an activated carboxyl group of 1,2-dihexadecanoyl-sn-glycero-3-phosphoethanolamine, and most preferably a glutaryl group of 1,2-dihexadecanoyl-sn-glycero-3-phosphoethanolamine-N-(glutaryl) sodium salt.

It is well known in the art how to covalently couple a protein to a carrier. It is preferred that that the carrier, in particular amphiphilic molecules forming the liposome, is covalently attached to the pathogen entry protein using a reagent selected from: carbodiimides, preferably N,N′-diisopropylcarbodiimide (DIC), N,N′-dicyclohexylcarbodiimide (DCC) or N-(3-Dimethylaminopropyl)-N′-ethylcarbodiimide hydrochloride (EDC); succinimidylesters, preferably sulfosuccinimide, N-hydroxybenzotriazole or N-hydroxysuccinimide (NETS); triazine-based coupling reagents, preferably 4-(4,6-Dimethoxy-1,3,5-triazin-2-yl)-4-methylmorpholiniumchloride (DMTMM); maleidesters; glutaraldehydecarbodiimide; and phosphonium or uronium based coupling agents, preferably benzotriazol-1-yloxytris(dimethylamino)phosphonium hexafluorophosphate (BOP), 1-Cyano-2-ethoxy-2-oxoethylidenaminooxy)dimethylamino-morpholino-carbenium hexafluorophosphate (COMU), 3-(diethoxyphosphoryloxy)-1,2,3-benzotriazin-4(3H)-one (DEPBT), 1-[Bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphate (HBTU), O-(1H-6-Chlorobenzotriazole-1-yl)-1,1,3,3-tetramethyluronium hexafluorophosphate (HCTU), benzotriazol-1-yl-oxytripyrrolidinophosphonium hexafluorophosphate (PyBOP), (7-Azabenzotriazol-1-yloxy)tripyrrolidinophosphonium hexafluorophosphate (PyAOP), (Ethyl cyano(hydroxyimino)acetato-O²)tri-1-pyrrolidinylphosphonium hexafluorophosphate (PyOxim) or O—(N-Succinimidyl)-1,1,3,3-tetramethyluronium tetrafluoroborate (TSTU).

In a further preferred embodiment, the carrier delivers or improves delivery of an antipathogenic agent to a target cell. Preferably, the target cell is a mammalian cell, more preferably a mammalian cell infected by a pathogen.

In another preferred embodiment the pathogen entry protein is an intracellular membrane protein from a bacterium, preferably from a Gram-negative bacteria. Typically, the pathogen entry protein is capable of interacting with an integrin receptor, preferably the β₁-integrin receptor. More preferably, the pathogen entry protein is capable of interacting with the extracellular domain of the β₁-integrin receptor. More preferably, the pathogen entry protein is a bacterial adhesion protein selected from invasin A, invasin B (Ifp), invasin C, invasin D, invasin E, YadA, other YadA-related (or YadA-type) proteins, internalin and fragments thereof. More preferably, the pathogen entry pathogen is invasin A or a fragment thereof.

The carrier system may comprise multiple carriers as described herein. Thus, typically, the carrier system comprises a single type of carrier. Alternatively, the carrier system comprises two or more types of carrier, e.g. two, three, four, five, six or more.

The carrier system may provide different forms of release kinetics according to the physical and chemical properties of the carrier. It is preferred that the release kinetic is selected from controlled release, preferably rapid release, delayed release, and sustained release. More preferably, the kinetic of the carrier systems is a sustained release kinetic. The API may be attached to the carrier either covalently or in a non-covalent manner, e.g. by van der Waals forces. In a preferred embodiment, the carrier system comprises the carrier and the pathogen entry protein covalently linked to one another, either directly or via a linker which may be straight or branched. In another preferred embodiment, the pathogen entry protein is linked either via its C-terminus, its N-terminus or a side chain, preferably the its N-terminus. It is noted that the C-terminus and N-terminus referred to in the context of the pathogen entry protein may be the natural C-terminus or N-terminus, but may also be the C-terminus or N-terminus that results when C-terminal or N-terminal amino acid sequences are removed from a naturally occurring pathogen entry protein.

Preferably, the pathogen entry protein is a protein or fragment thereof that is used by pathogenic organisms to enter a particular host cell of said pathogen and to infect said cell. Preferably, a chain of signalling cascades is provoked by the specific binding of said pathogen entry protein to a molecule on the surface of a target cell, leading to the rearrangement of the cytoskeletal system that leads to protrusions of the host membrane which surround the bacterium and internalizing it. It is preferred that said pathogen entry protein enters the cell via specifically binding to a molecule on the target cell's surface.

The fragment of the pathogen entry protein may be a contiguous part of the pathogen entry protein, shorter in length but having at least 70% sequence identity, preferably at least 75%, more preferably at least 80%, yet more preferably at least 85%, even more preferably at least 90%, and still more preferably least 95% sequence identity. It is preferred that the fragment also has the ability to specifically bind to a molecule on the surface of a mammalian target cell, which comprises a protein capable of specifically interacting with the pathogen entry protein. Preferably, the fragment consists or essentially consists of the extracellular domain of the pathogen entry protein. More preferably, the fragment consists or essentially consists of the extracellular domain and transmembrane domain of invasin. Even more preferably, the fragment consists or essentially consists of only the extracellular domain of invasin. Most preferably the fragment is encoded by SEQ ID NO: 2. The skilled person is well aware how to determine the extracellular domain of a given pathogen entry protein.

In another preferred aspect, the pathogen entry protein is an intracellular membrane protein from a bacterium, preferably from a Gram-negative bacterium. Even more preferably, it is from a bacterium that sequesters in a non-phagocytic cell.

In another preferred aspect, the pathogen entry protein is a bacterial adhesion protein selected from the group consisting of invasin A, invasin B (Ifp), invasin C, invasin D, invasin E, YadA, internalin and variants thereof. More preferably, the pathogen entry protein is invasin A. In a preferred embodiment, the pathogen entry protein has the amino acid sequence as indicated in SEQ ID NO: 1, or variants thereof with at least 70%, 75%, 80%, 85%, 90%, or 95% amino acid sequence identity and which specifically binds to the extracellular domain of the β₁-integrin receptor. In a further preferred embodiment, the pathogen entry protein has the amino acid sequence as indicated in SEQ ID NO: 2, or variants thereof with at least 70%, 75%, 80%, 85%, 90%, or 95% amino acid sequence identity and which specifically binds to the extracellular domain of the β₁-integrin receptor. In a further preferred embodiment, the pathogen entry protein has the amino acid sequence as indicated in SEQ ID NO: 3, or variants thereof with at least 70%, 75%, 80%, 85%, 90%, or 95% amino acid sequence identity and which specifically binds to the extracellular domain of the β₁-integrin receptor. In a further preferred embodiment, the pathogen entry protein has the amino acid sequence as indicated in SEQ ID NO: 4, or variants thereof with at least 70%, 75%, 80%, 85%, 90%, or 95% amino acid sequence identity and which specifically binds to the extracellular domain of the β₁-integrin receptor. In a further preferred embodiment, the pathogen entry protein has the amino acid sequence as indicated in SEQ ID NO: 5, or variants thereof with at least 70%, 75%, 80%, 85%, 90%, or 95% amino acid sequence identity and which specifically binds to the extracellular domain of the β_{ι}-integrin receptor. In a further preferred embodiment, the pathogen entry protein has the amino acid sequence as indicated in SEQ ID NO: 6, or variants thereof with at least 70%, 75%, 80%, 85%, 90%, or 95% amino acid sequence identity and which specifically binds to the extracellular domain of the β₁-integrin receptor. In a further preferred embodiment, the pathogen entry protein has the amino acid sequence as indicated in SEQ ID NO: 7, or variants thereof with at least 70%, 75%, 80%, 85%, 90%, or 95% amino acid sequence identity and which specifically binds to the extracellular domain of the β_{ι}-integrin receptor. In a further preferred embodiment, the pathogen entry protein has the amino acid sequence as indicated in SEQ ID NO: 8, or variants thereof with at least 70%, 75%, 80%, 85%, 90%, or 95% amino acid sequence identity and which specifically binds to the extracellular domain of the β₁-integrin receptor. In a preferred embodiment, the pathogen entry protein has the amino acid sequence as indicated in SEQ ID NO: 9, or variants thereof with at least 70%, 75%, 80%, 85%, 90%, or 95% amino acid sequence identity and which specifically binds to the extracellular domain of the β₁-integrin receptor.

Sequence identities between two proteins or nucleic acids are preferably determined over the entire length of the variant using the best sequence alignment with the reference sequence, e.g. SEQ ID NO: 1, and/or over the region of the best sequence alignment, wherein the best sequence alignment is obtainable with art known tools, e.g., Align, using standard settings, preferably EMBOSS:needle, Matrix:Blosum62, Gap Open 10.0, Gap Extend 0.5, with amino acid residues 1 to 210 of the amino acid sequence set forth in SEQ ID NO: 4. In another preferred embodiment, the fragment of the pathogen entry protein consists or essentially consists of the extracellular domain of the pathogen entry domain.

Typically, the molecule on the surface of the mammalian target cell provides specific binding of the pathogen entry protein. Preferably said molecule is selected from carbohydrates, lipids or proteins, and more preferably the molecule on the surface of the mammalian target cell is a protein. In a preferred embodiment the protein is capable of specifically interacting with the pathogen entry protein. It is preferred that the protein is a receptor protein which is usually found inside or on the surface of a cell that receives chemical signals from outside the cell. More preferably, the protein is selected from ionotropic receptors, kinase-linked and related receptors, nuclear receptors and G-protein coupled receptors. It is preferred that the protein is a member of the family of β-integrin receptors, and more preferably the protein is the β₁-integrin receptor. In another preferred embodiment, specific binding of the pathogen entry protein to the receptor protein causes some form of cellular/tissue response leading to the invasion of the pathogen entry protein into the mammalian target cell.

Typically, the pathogen is a microorganism selected from a virus, a bacterium, a prion, a fungus or a protozoan. Preferably, the pathogen is a bacteria selected from Gram-positive or Gram-negative bacteria. More preferably, the pathogen is a Gram-negative bacteria selected from Chlamydia, Coxiella burnetii, Ehrlichia, Rickettsia, Legionella, Salmonella, Shigella or Yersinia. Even more preferably the pathogen is Yersinia pseudotuberculosis or Yersinia enterocolitica.

Typically the mammalian target cell is any cell which originates from a mammal. It is preferred that the mammalian target cell is in an infected condition wherein this infected condition is triggered by a pathogen invaded in said mammalian cell. Preferably, said mammalian target cell is an endothelial cell or an epithelial cell. More preferably, said mammalian target cell is an epithelial cell.

The API or pharmaceutically acceptable salt can typically be any biologically active molecule, and is preferably at least one biologically active molecule selected from small molecule drugs, peptides, proteins, peptide mimetics, antibodies, antigens, deoxyribonucleic acid (DNA), messenger ribonucleic acid (mRNA), small interfering RNA, small hairpin RNA, microRNA, peptide nucleic acid (PNA), foldamers, carbohydrates, carbohydrate derivatives, non-Lipinski molecules, synthetic peptides synthetic oligonucleotides, and combinations thereof. Typically, a carrier system comprises a single API or pharmaceutically acceptable salt thereof. Alternatively, a carrier system may comprise two or more APIs or pharmaceutically acceptable salts thereof, e.g. two, three, four, five, six or more APIs or pharmaceutically acceptable salts thereof.

Typically, the pharmaceutically acceptable salt is selected from acetate, propionate, isobutyrate, benzoate, succinate, suberate, tartrate, citrate, fumarate, malonate, maleate, adipate, di-mesylate, sulfate, benzenesulfonate, nitrate, carbonate, hydrochloride, hydrobromide, phosphate, aluminium, ammonium, calcium, copper, ferric, ferrous, lithium, magnesium, manganic, manganous, potassium, sodium, zinc, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine and tromethamine salts of the API.

Typically, the API is a hydrophilic species. Alternatively, the API is a hydrophobic species. In some preferred embodiments, the API may be an anti-pathogenic agent, i.e. a species capable of either killing an infectious pathogen which invaded a host cell or decreasing the amount of infectious pathogen in a host cell invaded by said pathogen by interacting with the pathogen's molecular machinery. Preferably the anti-pathogenic agent is a hydrophilic anti-pathogenic agent, and is more preferably a small molecule, a protein, a nucleic acid (preferably siRNA), a nucleotide (preferably polynucleotide), an antibiotic or a cytostatic. Preferred antibiotics and cytostatics are described above. A suitable hydrophilic anti-pathogenic agent typically has a solubility of at least 10 g/mL.

The carrier system may be present within the film in varying amounts. Typically, the film comprises from 0.001% to 85% by weight of the carrier system, preferably from 0.01% to 75% by weight of the carrier system, and more preferably from 0.1% to 60% by weight of the carrier system.

The API may also therefore be present within the film in varying amounts. Typically, the film comprises from 0.0001% to 75% by weight of the API, preferably from 0.001% to 60% by weight of the API, more preferably from 0.01% to 50% by weight of the API, still more preferably from 0.1% to 45% by weight of the API and most preferably from 0.25% to 40% by weight of the API.

Preferably, a film of the present invention comprises from 15% to 99% by weight of the alginate salt of a monovalent cation or the mixture of alginate salts containing at least one alginate salt of a monovalent cation, from 0% to 20% by weight of water, and from 0.001% to 85% by weight of the carrier system. More preferably, the film comprises from 20% to 93% by weight of the alginate salt of a monovalent cation or the mixture of alginate salts containing at least one alginate salt of a monovalent cation, from 5% to 15% by weight of water, and from 0.01% to 75% by weight of the carrier system. Even more preferably, the film comprises from 25% to 91% by weight of the alginate salt of a monovalent cation or the mixture of alginate salts containing at least one alginate salt of a monovalent cation, from 9% to 11% by weight of water, and from 0.1% to 60% by weight of the carrier system.

A film according to the present invention may optionally further comprise other components in addition to those discussed above. Typically, a film according to the present invention further comprises one or more of the following:
(i) at least one pharmaceutically acceptable solvent;
(ii) at least one buffering component;
(iii) at least one excipient, such as one or more plasticizers, fillers, taste-masking agents or flavouring agents;
(iv) at least one acidifying agent or basifying agent;
(v) at least one permeation enhancer;
(vi) a self-emulsifying drug delivery system (SEDDS), such as a self-microemulsifying drug delivery system (SMEDDS) or a self-nanoemulsifying drug delivery system (SMEDDS);
(vii) at least one chelating agent;
(viii) at least one antioxidant;
(ix) at least one antimicrobial agent; and
(x) at least one inorganic salt.

The film may additionally comprise any pharmaceutically acceptable solvent. Such a solvent may be a non-aqueous solvent, or a combination of water and a non-aqueous solvent. Examples of non-aqueous solvents should be non-toxic and include, but are not limited to, ethanol, acetone, benzyl alcohol, diethylene glycol monoethyl ether, glycerine, hexylene glycol, isopropyl alcohol, polyethylene glycols, methoxypolyethylene glycols, diethyl sebacate, dimethyl isosorbide, propylene carbonate, dimethyl sulfoxide, transcutol, triacetin, fatty acid esters, and oils such as soybean oil, peanut oil, olive oil, palm oil, rapeseed oil, corn oil, coconut oil, other vegetable oils and the like.

The film may additionally comprise any suitable buffering component. A “buffering component”, as defined herein, refers to any chemical entity, which when dissolved in solution, enables said solution to resist changes in its pH following the subsequent addition of either an acid or a base. A suitable buffering component for use in the film of the present invention would be a buffering component which is an effective buffer within a pH range of from 3.0 to 5.5. Preferably, said buffering component is an effective buffer within a pH range of from 3.8 to 5.5. Examples of suitable buffering components include, but are not limited to: phosphates, sulfates, citrates and acetates. The buffer may be a salt of a monovalent cation, such as sodium, potassium or ammonium salts. Particularly preferred buffering components include citric acid and sodium dihydrogen phosphate. Without wishing to be bound by any particular theory, it is believed that alginate tends to gel at a pH of less than 3.8.

The film may comprise from 0.1% to 10% by weight of the buffering component, typically 0.2% to 8% by weight, typically from 0.3% to 6% by weight, typically from 0.5% to 5% by weight. Alternatively, the film may not additionally comprise a buffering component.

The film may additionally comprise any suitable excipient, such as one or more fillers or plasticizers. The film may comprise both a plasticizer and a filler. Alternatively, the film may comprise just one of a plasticizer or a filler. It is preferred that the film comprises a plasticizer. Under some circumstances it may be desirable that the film does not comprise a filler. It is particularly preferred that the film comprises a plasticizer but does not comprise a filler. The film may additionally include a taste-masking agent or a flavouring agent. The taste-masking agent may be a sweetener.

The plasticizer, when present, may be selected from polyethylene glycol, glycerol, sorbitol, xylitol, and a combination thereof. Typically, the film comprises a plasticizer which is selected from glycerol, sorbitol, xylitol, and a combination thereof. Preferably, the film comprises a plasticizer which is selected from glycerol, sorbitol, and a combination thereof. More preferably, the film comprises both glycerol and sorbitol as plasticizers. Most preferably, the film comprises glycerol, sorbitol and xylitol. The film may comprise from 0% to 40% by weight of each plasticizer present, preferably from 1% to 35% by weight of each plasticizer, more preferably from 2% to 30% by weight of each plasticizer, and most preferably from 3% to 25% by weight of each plasticizer. Without wishing to be bound by any particular theory, it is believed that the addition of plasticizers, e.g. a combination of glycerol, sorbitol and xylitol, increases the flexibility and pliability of the films, reducing brittleness. It is believed this makes the films easier to handle and use.

The filler, when present, may be e.g. microcrystalline cellulose or titanium dioxide. A suitable amount of filler may be from 0% to 20% by weight, e.g. from 0.1% to 10% by weight, of the total pharmaceutical composition.

The flavouring agent, when present, may for example be selected from acacia, anise oil, caraway oil, cardamom, cherry syrup, cinnamon, citric acid syrup, clove oil, cocoa, coriander oil, ethyl vanillin, fennel oil, ginger, glycerine, glycyrrhiza, honey, lavender oil, lemon oil, mannitol, nutmeg oil, orange oil, orange flower water, peppermint oil, raspberry, rose oil, rosewater, rosemary oil, sarsaparilla syrup, spearmint oil, thyme oil, tolu balsam syrup, vanilla, wild cherry syrup, and mixtures thereof. The film may comprise from 0.001% to 10% by weight of each flavouring agent present, preferably from 0.01% to 5% by weight of each flavouring agent, and most preferably from 0.1% to 3% by weight of each flavouring agent.

The film may additionally comprise an acidifying agent or a basifying agent. An “acidifying agent”, as defined herein, refers to a chemical compound that alone or in combination with other compounds can be used to acidify a pharmaceutical composition. A “basifying agent”, as defined herein, refers to a chemical compound that alone or in combination with other compounds can be used to basify a pharmaceutical composition.

Typically, the film comprises a basifying agent. Typically, the basifying agent is an alkali. Examples of suitable basifying agents include, but are not limited to: sodium hydroxide, lithium hydroxide, potassium hydroxide, magnesium hydroxide, and calcium hydroxide. A preferable basifying agent is sodium hydroxide. Alternatively, the film may comprise an acidifying agent. Examples of suitable acidifying agents include, but are not limited to: acetic acid, dehydro acetic acid, ascorbic acid, benzoic acid, boric acid, citric acid, edetic acid, hydrochloric acid, isostearic acid, lactic acid, nitric acid, oleic acid, phosphoric acid, sorbic acid, stearic acid, sulfuric acid, tartaric acid, and undecylenic acid. A preferable acidifying agent is phosphoric acid.

A film according to the present invention is produced via the drying of a film-forming solution (vide infra). Typically, a sufficient amount of acidifying agent or basifying agent is added to adjust the pH of the film-forming solution (before this is dried to form the film) to a pH of from 3.0 to 5.5, preferably to a pH of from 3.8 to 5.5.

The film may additionally comprise any suitable permeation enhancer. A “permeation enhancer”, as defined herein, refers to a chemical compound that alone or in combination with other compounds can be used to aid the uptake of a further substance across an epithelium or other biological membrane. In particular, the term “permeation enhancer” is used herein to refer to a chemical compound that alone or in combination with other compounds can be used to aid the uptake of a further substance across the buccal mucosa. Permeation enhancers can typically be divided into two different categories, paracellular (para) or transcellular (trans) permeability enhancers, according to their mechanism of action. Paracellular permeation enhancers are those which aid the uptake of a further substance through the intercellular space between the cells in an epithelium or other biological membrane. Transcellular permeation enhancers are those which aid the uptake of a further substance through the cells in an epithelium or other biological membrane, wherein the further substance passes through both the apical and basolateral cell membranes in the epithelium or other biological membrane.

Typically, the film may comprise one or more paracellular permeation enhancers. Alternatively, the film may comprise one or more transcellular permeation enhancers. Alternatively, the film may comprise at least one paracellular permeation enhancer and at least one transcellular permeation enhancer.

Typically, the permeation enhancer, if present, is one or more compounds selected from: non-ionic, cationic, anionic or zwitterionic surfactants (e.g. caprylocaproyl polyoxyl-8 glyceride, sodium lauryl sulfate, cetyltrimetyl ammonium bromide, decyldimethyl ammonio propane sulfonate); bile salts (e.g. sodium deoxycholate); fatty acids (e.g. hexanoic acid, hetptanoic acid, oleic acid); fatty amines; fatty ureas; fatty acid esters (e.g. methyl laurate, methyl palmitate); substituted or unsubstituted nitrogen-containing heterocyclic compounds (e.g. methyl pyrrolidone, methyl piperazine, azone); terpenes (e.g. limonene, fenchone, menthone, cineole); sulfoxides (e.g. dimethylsulfoxide, DMSO); ethylenediaminetetraacetic acid (EDTA); and combinations thereof. Preferably, the permeation enhancer, if present, is selected from EDTA, oleic acid, and combinations thereof.

Typically, the film may comprise EDTA. Without wishing to be bound by any particular theory, EDTA is believed to act as a paracellular permeation enhancer by transiently affecting tight junctions interconnecting membrane cells, and subsequently increasing paracellular or pore transport. EDTA is also believed to act as a transcellular permeation enhancer by interaction with phospholipid headgroups and increasing membrane fluidity [3]. Alternatively, the film may comprise oleic acid. Without wishing to be bound by any particular theory, oleic acid is believed to act as a transcellular permeation enhancer by interacting with the polar head groups of phospholipids in or on cell membranes, and increasing cell membrane flexibility, thereby promoting transcellular drug permeability. Oleic acid has been shown to demonstrate enhanced permeability with porcine buccal epithelium at a concentration of 1-10% [4].

The film may additionally comprise a self-emulsifying drug delivery system (SEDDS) or resulting emulsion thereof. Such a system may preferably be a self-microemulsifying drug delivery system (SMEDDS) or resulting emulsion thereof or a self-nanoemulsifying drug delivery system (SNEDDS) or resulting emulsion thereof. Self-microemulsifying drug delivery systems are microemulsion preconcentrates or anhydrous forms of microemulsion. Self-nanoemulsifying drug delivery systems are nanoemulsion preconcentrates or anhydrous forms of nanoemulsion. These systems are typically anhydrous isotropic mixtures of oil (e.g. tri-, di- or mono-glycerides or mixtures thereof) and at least one surfactant (e.g. Span®, Tween®), which, when introduced into aqueous phase under conditions of gentle agitation, spontaneously form an oil-in-water (O/W) microemulsion or nanoemulsion (respectively). SNEDDS systems typically form an emulsion with a globule size less than 200 nm [5]. SEDDS (e.g. SMEDDS or SNEDDS) may also contain coemulsifier or cosurfactant and/or solubilizer in order to facilitate emulsification (e.g. micoremulsification or nanoemulsification) or improve the drug incorporation into the SEDDS (e.g. SMEDDS or SNEDDS).

Optionally, the oil phase is selected from olive oil, soyabean oil, Capryol PGMC, Maisine CC, Labrafil M2125, Captex 355 and triacetin, preferably Capryol PGMC. Optionally, the at least one surfactant is selected from Cremophor EL, Tween 80 and Labrasol. The SEDDS may comprise at least two surfactants, preferably wherein said surfactants are selected from Cremophor EL, Tween 80 and Labrasol. For example, the SEDDS may comprise both Cremophor EL and Labrasol as surfactants. In some embodiments, the SEDDS further comprises a solubilizer (cosolvent). Typical solubilizers include transcutol, polyethylene glycol (PEG), DMSO and ethanol. A particularly preferred solubilizer is transcutol.

Typically, the SEDDS (e.g. SMEDDS or SNEDDS) components is selected from the group consisting of: a mixture of Tween® with one or more glycerides and a hydrophilic cosolvent; a mixture of Tween® with a low HLB (hydrophile-lipophile balance) cosurfactant and a hydrophilic cosolvent; a mixture of a polyethyleneglycol (PEG), Labrasol and Chremophore EL; a mixture of polyethyleneglycol (PEG), Labrasol and Kolliphore EL; a mixture of polyethyleneglycol (PEG), Labrasol, Chremophore EL and Chremophore RH40; a mixture of Capryol PGMC, Cremophor EL and transcutol; a mixture of Capryol PGMC, Cremophor EL and Labrasol; and a mixture of Capryol PGMC, Cremophor EL, Labrasol and transcutol. The PEG may be any suitable polyethyleneglycol such as PEG with an average molecular weight of from 100 to >1000 Da, preferably from 200 to 800 Da, more preferably from 300 to 600 Da, and most preferably about 400. More preferably, the SEDDS components is selected from the group consisting of: a mixture of Capryol PGMC, Cremophor EL and transcutol; a mixture of Capryol PGMC, Cremophor EL and Labrasol; and a mixture of Capryol PGMC, Cremophor EL, Labrasol and transcutol.

The term “glyceride”, as defined herein, refers to any ester formed between glycerol and one or more fatty acids. The term “glyceride” may be used interchangeably with the term “acylglycerol”. Typically, the glyceride is a monoglyceride, a diglyceride or a triglyceride. Preferably, the glyceride is a triglyceride. Typically, the glyceride is a simple glyceride. The term “simple glyceride” refers to a diglyceride in which the two fatty acids are the same as one another, or a triglyceride in which the three fatty acids are the same as one another. Alternatively, the glyceride is a mixed glyceride. The term “mixed glyceride” refers to a diglyceride in which the two fatty acids are different one another, or a triglyceride in which either one of the three fatty acids is different to the other two, or all three of the fatty acids are different to one another. Therefore, the glyceride is typically a monoglyceride, a simple diglyceride, a simple triglyceride, a mixed diglyceride, or a mixed triglyceride. Preferably, the glyceride is a simple triglyceride or a mixed triglyceride.

A “hydrophilic cosolvent”, as defined herein, is any solvent that is miscible with water. Examples of suitable hydrophilic cosolvents include, but are not limited to: glycerol, ethanol, 2-(2-ethoxyethoxyethanol), PEG-400 and propylene glycol.

The term “low HLB cosurfactant”, as defined herein, refers to any lipid falling within class IIIA, IIIB or IV of the lipid formulation classification system described by C. W. Pouton [6], the contents of which are herein incorporated by reference in their entirety.

Typically, the film may additionally comprise any suitable chelating agent. A chelating agent may be added to the film to act as a preservative. A “chelating agent”, as defined herein, refers to a chemical compound that is a multidentate ligand that is capable of forming two or more separate bonds to a single central atom, typically a metal ion. Examples of suitable chelating agents include, but are not limited to: ethylenediaminetetraacetic acid (EDTA), ethylene glycol-bis(β-aminoethyl ether)-N,N,N′,N′-tetraacetic acid (EGTA), 1,2-bis(ortho-aminophenoxy)ethane-N,N,N′,N′-tetraacetic acid (BAPTA), citric acid, phosphonic acid, glutamic acid, histidine, malate, and derivatives thereof. Preferably, the chelating agent, if present, is ethylenediaminetetraacetic acid (EDTA). The film may comprise from 0.001% to 4% by weight of each chelating agent present. Preferably, the film may comprise from 0.001% to 0.1% by weight of each chelating agent present.

The film may additionally comprise any suitable antioxidant. An “antioxidant”, as defined herein, is any compound that inhibits the oxidation of other chemical species. Examples of suitable antioxidants include, but are not limited to: ascorbic acid; citric acid; sodium bisulfite; sodium metabisulfite; ethylenediaminetetraacetic acid (EDTA); butyl hydroxitoluene; and combinations thereof. Preferably, the antioxidant, if present, is ascorbic acid, sodium bisulfite, or a combination thereof. More preferably, the antioxidant, if present, is ascorbic acid. Most preferably, both ascorbic acid and sodium bisulfite are present as antioxidants. Preferably, the film may comprise from 0.001% to 4% by weight of each antioxidant present, more preferably from 0.001% to 0.1% by weight of each antioxidant present.

Typically, the film may additionally comprise any suitable antimicrobial agent. An “antimicrobial agent”, as defined herein, is any compound that kills microorganisms or prevents their growth. Examples of suitable antimicrobial agents include, but are not limited to: benzyl alcohol; benzalkonium chloride; benzoic acid; methyl-, ethyl- or propyl-paraben; and quarternary ammonium compounds. The film may comprise from 0.001% to 4% by weight of each antimicrobial agent present. Preferably, the film may comprise from 0.001% to 0.1% by weight of each antimicrobial agent present.

EDTA may therefore be present in a film according to the present invention as an antioxidant, as a permeation enhancer or as a chelating agent. Typically, if EDTA is present, the EDTA acts as all of an antioxidant, a permeation enhancer and a chelating agent. Alternatively, if EDTA is present, the EDTA may act only as an antioxidant. Alternatively, if EDTA is present, the EDTA may act only as a permeation enhancer. Alternatively, if EDTA is present, the EDTA may act only as a chelating agent.

Optionally, the film may additionally comprise at least one inorganic salt. Said inorganic salt may be any salt acceptable for use in the preparation of a medicament. Examples of such salts include, but are not limited to, the halides, oxides, hydroxides, sulfates, carbonates, phosphates, nitrates, acetates and oxamates of the alkali metals, alkaline earth metals, aluminium, zinc and ammonium. Typically, said inorganic salt may be selected from sodium chloride, potassium chloride, magnesium chloride, calcium chloride, and ammonium chloride. Preferably, the inorganic salt is sodium chloride. Typically, the inorganic salt is present in the film in a total concentration of at least 0.05 wt %, preferably in a concentration of from 0.1 to 5 wt %, more preferably from 0.2 to 2 wt %, yet more preferably from 0.25 to 1 wt %, and most preferably about 0.5 wt %. Alternatively, the film does not comprise any inorganic salt. In such an embodiment, the film typically comprises the neutral (i.e. unionized) form of the API.

Typically, the film may additionally comprise at least one excipient, optionally at least one basifying agent or acidifying agent, optionally at least one permeation enhancer, optionally at least one pharmaceutically acceptable solvent, optionally at least one buffering component, optionally at least one antioxidant, and optionally a SEDDS (e.g. SMEDDS or SMEDDS). For example, the film may comprise at least one excipient, at least one basifying agent or acidifying agent, optionally at least one permeation enhancer, optionally at least one anitoxidant and optionally at least one buffering component. Preferably, the film may comprise glycerol, sorbitol, optionally at least one basifying agent or acidifying agent, optionally at least one permeation enhancer, optionally at least one antioxidant, and optionally at least one buffering component. For example, the film may comprise glycerol, sorbitol and xylitol.

Preferably, the film according to the present invention comprises from 15% to 99% by weight of the alginate salt of a monovalent cation or the mixture of alginate salts containing at least one alginate salt of a monovalent cation, from 0% to 20% by weight of water, from 0.001% to 85% by weight of the carrier system, from 0.0001% to 75% by weight of the API, from 0% to 40% by weight of glycerol, from 0% to 40% by weight of sorbitol, optionally from 0% to 40% by weight of xylitol, optionally a basifying agent or an acidifying agent, optionally from 0.01% to 5% by weight of a permeation enhancer, optionally from 0.01% to 10% by weight of at least one antioxidant, optionally from 0.1% to 10% by weight of a SEDDS (e.g. SMEDDS or SNEDDS), and optionally from 0.001% to 4% by weight of a chelating agent. More preferably, the film according to the present invention comprises from 30% to 80% by weight of the alginate salt of a monovalent cation or the mixture of alginate salts containing at least one alginate salt of a monovalent cation, from 9% to 11% by weight of water, from 0.1% to 60% by weight of the carrier system, from 0.01% to 50% by weight of the API, from 5% to 20% by weight of glycerol, from 5% to 20% by weight of sorbitol, optionally from 5% to 20% by weight of xylitol, and optionally a basifying agent or an acidifying agent.

Alternatively, the film according to the present invention consists of from 15% to 99% by weight of the alginate salt of a monovalent cation or the mixture of alginate salts containing at least one alginate salt of a monovalent cation, from 0% to 20% by weight of water, from 0.001% to 85% by weight of the carrier system, from 0.0001% to 75% by weight of the API, from 0% to 40% by weight of glycerol, from 0% to 40% by weight of sorbitol, optionally from 0% to 40% by weight of xylitol, optionally a basifying agent or an acidifying agent, optionally from 0.01% to 5% by weight of a permeation enhancer, optionally from 0.01% to 10% by weight of at least one antioxidant, optionally from 0.1% to 10% by weight of a SEDDS (e.g. SMEDDS or SNEDDS), and optionally from 0.001% to 4% by weight of a chelating agent. More preferably, the film according to the present invention consists of from 30% to 80% by weight of the alginate salt of a monovalent cation or the mixture of alginate salts containing at least one alginate salt of a monovalent cation, from 9% to 11% by weight of water, from 0.1% to 60% by weight of the carrier system, from 0.001% to 50% by weight of the API, from 5% to 20% by weight of glycerol, from 5% to 20% by weight of sorbitol, optionally from 5% to 20% by weight of xylitol, and optionally a basifying agent or an acidifying agent.

A film according to the invention preferably has a thickness before drying of 200 to 2000 μm, more preferably from 300 to 1750 μm, even more preferably from 400 to 1500 μm, and most preferably from 1000 to 1200 μm.

A film according to the invention preferably has a surface area on each of its two largest faces of from 0.1 to 20 cm², more preferably from 0.5 to 15 cm², even more preferably from 1 to 10 cm² and most preferably from 2 to 6 cm². Preferably, the surface area of each of the two largest faces of the film is about 3 cm² or about 5 cm².

The skilled person, having regard for the desired time of dissolution for a given application, will be able to select a suitable film thickness and surface area by simply preparing films of a range of different thicknesses and surface areas and testing the resultant films to measure the dissolution time.

The mechanical properties of a film according to the invention are very satisfactory. In particular, the film is flexible (i.e. it permits bending and folding without breaking), and has a high tensile strength. Importantly, the film of the present invention is not a gel, since the alginate polymer strands are not cross-linked with one another. The film of the invention is bioadhesive; that is to say that the film comprises a natural polymeric material (alginate) which can act as an adhesive. The film is adhesive to moist surfaces, such as mucosa. In particular, the film is adhesive to mucosa of the oral cavity, such as mucosa in the buccal, labial, sublingual, ginigival or lip areas, the soft palate and the hard palate.

The film according to the invention may be provided with printed text matter or printed images thereon, e.g. a brand name, a trade mark, a dosage indication or a symbol.

### Administration and Uses of the Films in Treatment

In general, films of the present invention are administered to a human patients so as to deliver to the patient a therapeutically effective amount of the active pharmaceutical ingredient (API) or pharmaceutically acceptable salt thereof contained therein.

As used herein, the term “therapeutically effective amount” refers to an amount of the API which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of API administered to a patient will depend on the type and severity of the disease or condition and on the characteristics of the patient, such as general health, age, sex, body weight and tolerance to drugs. It will also depend on the degree, severity and type of the disorder being treated. The skilled artisan will be able to determine appropriate dosages depending on these and other factors.

As used herein, the terms “treat”, “treatment” and “treating” refer to the reduction or amelioration of the progression, severity and/or duration of a disorder being treated, or the amelioration of one or more symptoms (preferably, one or more discernible symptoms) of a disorder being treated resulting from the administration of a film according to the invention to a patient.

Typically, a film according to the present invention is provided for use in the treatment of a human patient. Typically, the film according to the invention is provided for use in the treatment or prophylaxis of infectious disease in a human patient. Alternatively, the film according to the invention is provided for use in the treatment or prophylaxis of a disease or condition selected from diabetes mellitus, insulinoma, metabolic syndrome and polycysic ovary syndrome in a human patient.

A film according to the invention may be provided for use in the treatment of a systemic infection, preferably nosocomial infections, more preferably elicited by Staphylococcus and/or vancomycin-resistant Enterococcus (VRE). In another preferred embodiment the infectious disease is an infection with a bacterium, which persists/replicates (sequesters) in non-phagocytic cells, preferably a Gram-negative bacterium, more preferably Chlamydia, Coxiella burnetii, Ehrlichia, Rickettsia, Legionalla, Salmonella, Shigella or Yersinia, or a Gram-positive bacterium, more preferably Mycobacterium leprae or Mycobacterium tuberculosis.

Other infections that can be treated with the films of the present invention include Leprosy, Leishmaniasis, Malaria, Tuberculosis, Dengue and severe dengue, Buruli ulcer, Hepatitis B, Hepatitis E, Hepatitis C, Hepatitis A, Trypanosomiasis, Human African (sleeping sickness), Poliomyelitis, Measles, Crimean-Congo haemorrhagic fever, Meningococcal meningitis, Ebola haemorrhagic fever, Cholera, Monkeypox, Influenza, Rift Valley fever, and Smallpox.

A film according to the present invention may alternatively be provided for use in the treatment of a disease or condition selected from diabetes mellitus, insulinoma, metabolic syndrome and polycysic ovary syndrome. Typically, in such a film, the API is insulin or a derivative thereof, optionally in combination with one or more other active agents. Preferably, in such a film the API is insulin.

Typically, the patient to be treated is an adult. Alternatively, the patient to be treated may be a child. The patient to be treated may be an elderly patient. The patient to be treated may be a child suffering from allergies.

Typically, the film is administered to the oral cavity of the patient. The film is preferably applied to an oral mucosa in the buccal or labial or sublingual areas or to the soft palate. The film is typically applied by the patient themselves. Alternatively, the film is administered to the patient by another person, e.g. a medical practitioner, a nurse, a carer, a social worker, a colleague of the patient or a family member of the patient.

The film is bioadhesive and adheres to the surface of the oral cavity upon application. After application, the alginate film begins to dissolve, releasing the active pharmaceutical ingredient. Typically, the film fully dissolves in a time period of from 0.1 to 60 minutes or more after application to the mucosa of the oral cavity. Preferably, the film fully dissolves in a time period of from 0.5 to 30 minutes, more preferably from 1 to 20 minutes, still more preferably from 3 to 10 minutes, and most preferably from 3 to 5 minutes after application to the mucosa of the oral cavity.

Without wishing to be bound by any particular theory, it is believed that as the film dissolves within the oral cavity, the active pharmaceutical ingredient which is concomitantly released may enter the bloodstream by one or both of two different routes: (a) via absorption across the oral mucosa directly into the bloodstream (the “oral transmucosal route”); and (b) via swallowing into the stomach and subsequent absorption across the epithelium of the intestines into the bloodstream. Typically the peak plasma concentration of the API in a patient exceeds 0.01 ng/mL, and more preferably exceeds 0.1 ng/mL. This peak plasma concentration may be achieved within 8 hours from adhesion of the film to the mucosa of the oral cavity, preferably within 6 hours from adhesion, and more preferably within 4 hours from adhesion.

Typically, a single film is applied to the patient, generally to the mucosa of the oral cavity, at a given time. However, in some cases it may be desirable to apply two films simultaneously to achieve the correct dose for an individual patient. In some cases it may be desirable to apply more than two films simultaneously to achieve the correct dose for an individual patient, for example, three, four, five, six, seven, eight, nine, ten or more.

The present invention also therefore provides a method of treating infectious disease in a human patient, wherein said method comprises administration of at least one film according to the invention to a human patient.

The present invention also provides the use of a film according to the invention for the manufacture of a medicament for treating infectious disease in a human patient.

The present invention also provides a product comprising one or more films according to the invention, and packaging. Each of the films may individually be wrapped within a pouch, or multiple films may be wrapped together within the same pouch. Optionally, said pouch is made from PET-lined aluminium. The product may further comprise instructions for use of the film. These instructions may contain information on the recommended frequency or timing of use of the film by a patient, how to use remove the film from its pouch or packaging, how to adhere the film to a mucous membrane, and where within the oral cavity to adhere the film to a mucous membrane.

Any film or films of the present invention may also be used in combination with one or more other drugs or pharmaceutical compositions in the treatment of disease or conditions for which the films of the present invention and/or the other drugs or pharmaceutical compositions may have utility.

The one or more other drugs or pharmaceutical compositions may be administered to the patient by any one or more of the following routes: oral, systemic (e.g. transdermal, intranasal, transmucosal or by suppository), or parenteral (e.g. intramuscular, intravenous or subcutaneous). Compositions of the one or more other drugs or pharmaceutical compositions can take the form of tablets, pills, capsules, semisolids, powders, sustained release formulations, solutions, suspensions, elixirs, aerosols, transdermal patches, bioadhesive films, or any other appropriate compositions. The choice of formulation depends on various factors such as the mode of drug administration (e.g. for oral administration, formulations in the form of tablets, pills or capsules are preferred) and the bioavailability of the drug substance.

### Manufacture of the Films

The films according to the invention may be manufactured by preparing a film-forming solution by addition and mixing of the constituent components of the film, distributing this solution onto a solid surface, and permitting the solution to dry on the surface to form a film. To distribute a solution or composition onto a solid surface the solution or composition may simply be poured onto and/or spread evenly over the surface, e.g. by use of a draw-down blade or similar equipment.

A typical method includes the process steps of:
(a) covalently linking a pathogen entry protein or part thereof to a carrier either prior or after contacting the carrier with at least one API or a pharmaceutically acceptable salt thereof, to form a carrier system;
(b) mixing the carrier in water, and optionally subsequently adjusting the pH of the solution to the desired level by addition of an appropriate acid or base, typically a concentrated acid, and preferably adjusting the pH of the solution to from 2 to 4;
(c) optionally, mixing one or more excipients into the solution;
(d) adding the alginate salt of monovalent cation under suitable conditions to result in the formation of a viscous cast;
(e) adjusting the pH of the solution to the desired level by addition of an appropriate acid or base, typically a diluted acid or alkali, preferably a diluted alkali, and preferably adjusting the pH of the solution to from 3 to 5;
(f) optionally, sonicating the cast;
(g) leaving the cast to de-aerate;
(h) pouring the cast onto a surface and spreading the cast out to the desired thickness;
(i) drying the cast layer, typically at a temperature of from −10 to 30° C., preferably from 0 to 10° C., and more preferably from 4 to 8° C., and typically at a pressure of from 0.2 atm to 1 atm, preferably from 0.4 to 0.95 atm, until the residual water content of the film is from 0 to 20% by weight, preferably from 5 to 15% by weight, and more preferably from 9 to 11% by weight, and a solid film is formed; and
(j) optionally, cutting the solid film into pieces of the desired size, further optionally placing these pieces into pouches, preferably wherein the pouches are made from PET-lined aluminium, sealing the pouches and further optionally, labelling them.

In a preferred embodiment, any one or any combination of steps (b) to (h) are carried out at a temperature of from −10 to 30° C., preferably from 0 to 10° C., and more preferably from 4 to 8° C.

When the films are to be formulated as emulsion-based films, an alternative method for manufacturing a film according to the invention that is particularly preferred includes the process steps of:
(a) covalently linking a pathogen entry protein or part thereof to a carrier either prior or after contacting the carrier with at least one API or a pharmaceutically acceptable salt thereof, to form a carrier system;
(b) mixing the carrier in an oil phase;
(c) premixing a surfactant and a cosolvent, and then adding this to the solution obtained in step (b) under mixing;
(d) optionally, adding one or more excipients, flavouring agents, buffering components, permeation enhancers, chelating agents, antioxidants and/or antimicrobial agents to water;
(e) adding water, or the solution obtained in step (d), to the solution obtained in step (c) under stirring, preferably continuous stirring, and more preferably wherein the water or the solution obtained in step (d) is added in a dropwise fashion;
(f) optionally, storing the solution obtained in step (e) overnight and subsequently evaluating its physical stability;
(g) mixing the alginate salt of monovalent cation in the solution, until a lump free dispersion is achieved, and optionally adding further water to modulate the viscosity of the cast formed;
(h) pouring the cast onto a surface, e.g. a plate, preferably a glass plate, and spreading the cast out to the desired thickness, e.g. about 1 mm, or about 1.2 mm if further water was added in step (g), typically by means of an applicator;
(i) drying the cast layer, typically at a temperature of from −10 to 30° C., preferably from 0 to 10° C., and more preferably from 4 to 8° C., and typically at a pressure of from 0.2 atm to 1 atm, preferably from 0.4 to 0.95 atm, until the residual water content of the film is from 0 to 20% by weight, preferably from 5 to 15% by weight, and more preferably from 9 to 11% by weight; and
(j) optionally, cutting the solid film into pieces of the desired size, further optionally placing these pieces into pouches, preferably wherein the pouches are made from PET-lined aluminium, sealing the pouches and further optionally, labelling them.

In a preferred embodiment, any one or any combination of steps (b) to (h) are carried out at a temperature of from −10 to 30° C., preferably from 0 to 10° C., and more preferably from 4 to 8° C.

In step (a) of any of the above methods, the contacting of the carrier with the at least one API or a pharmaceutically acceptable salt thereof serves the purpose of loading the API into or onto the carrier. Hydrophilic APIs can be passively loaded into liposomes during the preparation process by using an aqueous solution containing the hydrophilic API as hydrating medium. Passive loading of drugs can be achieved by a number of different techniques, including mechanical dispersion methods, solvent dispersion methods and detergent removal methods, as mentioned below.

The mechanical dispersion method (MDM) involves two main steps: drying of lipids dissolved in an organic solvent, followed by mechanical dispersion of these dry lipids in an aqueous medium. In most cases, this is achieved by shaking. A hydrophilic API can be incorporated into the aqueous medium, while a hydrophobic/lipophilic API is dissolved together with lipids in the organic solvent. At this stage, various techniques can be used to modify the formed liposomes depending on the desired vesicle type and size. Sonication can be used to prepare SUVs, while extrusion can be used to prepare LUVs large unilamellar vesicles. The MLVs multilamellar vesicles can be prepared using techniques such as the freeze-thaw method or the sonicate-dehydrate-rehydrate method. In the solvent dispersion method (SDM) lipids are first dissolved in an organic solvent, and then mixed with an aqueous medium, hydrophobic drug is dissolved with the lipids into the organic solvent and hydrophilic API is dissolved in the aqueous medium, using two techniques to form liposomes. The ethanol injection technique involves a direct and rapid injection of lipids dissolved in ethanol to an aqueous medium through a fine needle. The ether injection technique involves a careful and slower injection of this immiscible organic solvent containing the lipid into an aqueous medium containing the drug at high temperature. The detergent removal method involves the use of intermediary detergents in the lipid dispersion phase, such as cholate, alkyl-glycoside or Triton X-100. This detergent then associates with lipids to solubilize them and form micelles. In order to transform micelles into liposomes, the detergent must be removed. The removal of the detergent can be achieved by different techniques such as dialysis or gel chromatography. Active loading of some chemical molecules such as lipophilic ions and weak acids and bases into liposomes can be achieved by various transmembrane gradients, including electrical gradients, ionic gradients or chemical potential gradients. All these concepts follow one principle that the free drug diffuses through the liposome. The diffusion requires two modification steps; one allows the drug to enter and the second inhibits membrane re-permeation resulting in drug accumulation inside liposomes. Weak bases like doxorubicin and vincristine which coexist in aqueous solutions in neutral and charged forms have been successfully loaded into performed liposomes via the pH gradient method. Other approaches have also been employed in which an ammonium sulfate gradient or calcium acetate gradient are used as the driving force for loading of amphipathic drugs.

In a preferred embodiment, the pathogen entry protein and/or at least one constituent of the carrier comprises an activatable group prior to covalent linking. Preferably said activatable group is activated with an activating reagent selected from: carbodiimides, preferably N,N′-diisopropylcarbodiimide (DIC), N,N′-dicyclohexylcarbodiimide (DCC) or N-(3-Dimethylaminopropyl)-N′-ethylcarbodiimide hydrochloride (EDC); succinimidylesters, preferably sulfosuccinimide, N-hydroxybenzotriazole or N-hydroxysuccinimid (NETS); triazine-based coupling reagents, preferably 4-(4,6-Dimethoxy-1,3,5-triazin-2-yl)-4-methylmorpholiniumchloride (DMTMM); maleidesters; glutaraldehyde; and phosphonium or uronium based coupling agents, preferably benzotriazol-1-yloxytris(dimethylamino)phosphonium hexafluorophosphate (BOP), 1-Cyano-2-ethoxy-2-oxoethylidenaminooxy)dimethylamino-morpholino-carbenium hexafluorophosphate (COMU), 3-(diethoxyphosphoryloxy)-1,2,3-benzotriazin-4(3H)-one (DEPBT), 1-[Bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphate (HBTU), O-(1H-6-Chlorobenzotriazole-1-yl)-1,1,3,3-tetramethyluronium hexafluorophosphate (HCTU), benzotriazol-1-yl-oxytripyrrolidinophosphonium hexafluorophosphate (PyBOP), (7-Azabenzotriazol-1-yloxy)tripyrrolidinophosphonium hexafluorophosphate (PyAOP), (Ethyl cyano(hydroxyimino)acetato-O²)tri-1-pyrrolidinylphosphonium hexafluorophosphate (PyOxim) or O—(N-Succinimidyl)-1,1,3,3-tetramethyluronium tetrafluoroborate (TSTU). More preferably, the activatable group is a carbodiimide or a succinimidylester. Most preferably, the activating reagent is a mixture of N-(3-dimethylaminopropyl)-N′-ethylcarbodiimide hydrochloride (EDC) and N-hydroxysuccinimide (NETS), preferably wherein EDC is at a concentration of from 5 to 100 mM, more preferably from 20 to 60 mM, and NETS is at a molar concentration of from 1 to 50 mM, more preferably from 10 to 30 mM.

In an alternative variant of any of the above methods, after the viscous cast is poured onto a surface, it is first spread out to a thickness of about 2 mm by means of an applicator with a slit height of about 2 mm, and is then subsequently spread out to a thickness of about 1 mm by means of an applicator with a slit height of about 1 mm.

Typically, the alginate salt(s) are added to the carrier system-containing water solution. Alternatively, the carrier system and the alginate salt(s) are both dissolved together in solution. Alternatively, the carrier system may be added to the alginate solution so as to give an emulsion or suspension of the carrier system in the alginate solution. Alternatively, the film-forming composition of the invention may comprise both dissolved and non-dissolved active ingredients. For example, a film-forming composition may comprise a combination of active ingredient dissolved in the alginate solution and active ingredient suspended in the solution.

Additional carrier system may be applied to the surface of the film before or after drying, e.g. as an aerosol spray onto a dry or wet film. An active ingredient may also be applied as a powder onto the surface of the film. A flavouring agent may additionally be applied in such a way.

The publications, patent publications and other patent documents cited herein are entirely incorporated by reference. Herein, any reference to a term in the singular also encompasses its plural. Where the term “comprising”, “comprise” or “comprises” is used, said term may substituted by “consisting of”, “consist of” or “consists of” respectively, or by “consisting essentially of”, “consist essentially of” or “consists essentially of” respectively. Any reference to a numerical range or single numerical value also includes values that are about that range or single value. Any reference to alginate encompasses any physiologically acceptable salt thereof unless otherwise indicated. Unless otherwise indicated, any % value is based on the relative weight of the component or components in question.

### EXAMPLES

The following are Examples that illustrate the present invention. However, these Examples are in no way intended to limit the scope of the invention.

### Example 1: Preparation of Films Comprising Gentamicin-Containing Liposomes

Liposomes containing gentamicin as active agent are prepared and functionalised with invasin A497 as described in WO 2016/024008.

In order to prepare alginate-containing films comprising these liposomes, a self-emulsifying mixture is used to aid the solubility of the liposomes. In a test formulation, a SEDDS formulation containing capryol PGMC (which consists of propylene glycol mono- and di-esters of caprylic acid) as oil phase and transcutol (highly purified diethylene glycol monoethyl ether) as cosolvent, is selected. Surfactants displaying higher HLB values such as the polyoxyethylene caster oil derivative Cremophor EL (13.9) and Tween 80 are selected. Capryol PGMC (oil), Cremophor EL and Tween 80 (surfactants) and transcutol (cosolvent) were thus used to prepare a microemulsion for dissolving the gentamicin-containing liposomes (see Table 1 below). The mass ratio of surfactant to cosolvent (Smix ratio) is kept constant at 1:1 by weight.

The use of transcutol as a cosolvent is believed to contribute to the formation of emulsion/microemulsion by multiple mechanisms such as through reducing interfacial tension and viscosity, with the cosolvent molecules positioning themselves in-between the surfactant tails and thus increasing the flexibility and fluidity of the interfacial film.

TABLE 1
Composition of SMEDDS with the basic formulation
for 5 mg gentamicin films.

| | | | Concentration | |
|---|---|---|---|---|
| Ingredient | Amount | | (w/w) | Function |
| Capryol PGMC | 1.5 | g | 3% | Oil |
| Cremophor EL/ | 2.5 | g | 5% | Surfactant |
| Tween 80 | | | | |
| Transcutol | 2.5 | g | 5% | Cosolvent |
| NaCl | 0.25 | g | 0.5% | Salt |
| milliQ Water | 50 | mL | | Solvent |
| Glycerol | 1.5 | g | | Plasticizer |
| Sorbitol | 1.75 | g | | Plasticizer |
| Sodium alginate | 6.65 | g | | Film-Forming |
| (Protanal 5/60) | | | | Polymer |

The batch formula for a film preparation containing 5 mg gentamicin/dose is as set out in Table 1 above.

The films are prepared as follows:
The liposomes are solubilized in 3% w/w of oil phase (capryol PGMC) and the surfactants and cosolvent are added under continuous stirring.
The milliQ water is added to the lipid mixture in dropwise manner.
These emulsions are stored overnight and later, subjected to visual assessment of physical stability (i.e. presence of coalescence or phase separation).
Sodium chloride, glycerol and sorbitol are added to the solution/emulsion.
The sodium alginate is added to the solution/emulsion under mixing until a lump free and smooth solution/emulsion was achieved.
The cast is left overnight for de-aeration.
The cast is poured onto a glass plate and spread out to a thickness of 1 mm by means of an applicator.
The cast layer is dried at a temperature of about 5° C. and about 0.2 or about 0.4 or about 0.6 atm pressure until a residual water content of from 9% to 11% by weight is achieved and a solid film is formed.
The solid film is cut into pieces measuring 20×30 mm with a knife.
The resulting films are placed individually into PET-lined aluminium pouches, sealed with a heat sealer and labelled.

### Example 2: Physical Evaluation of Carrier-Containing Films

After manufacture, each of the batches of carrier-containing films may be evaluated with respect to the following criteria:

| Property | | Criteria |
|---|---|---|
| 1. | Cast texture: | lump free, homogenous viscous cast |
| | | (visual inspection) free of bubbles |
| | | prior to coating (visual inspection) |
| 2. | Residual moisture*: | 9-11% (in process control) |
| 3. | Film appearance: | translucent and homogenous (visual inspection) |
| | | smooth and flat surface structure (visual |
| | | inspection) |
| | | pliable and flexible (visual inspection) |
| 4. | Dose weight | weighing of doses randomly selected |
| | homogeneity: | within a film batch |
| 5. | Gentamicin | target dose strength within ±10% |
| | content: | by weight (RP-HPLC analysis) |
| 6. | Physical stability | oil release (visual inspection) |
| | | crystal free film (optical microscopy study) |

### REFERENCES

[1] WO 2016/024008.
[2] He et al., Adapating liposomes for oral drug delivery. Acta Pharmaceutica Sinica B, 2019, 36-48
[3] Prachayasittikul, V.; Isarankura-Na-Ayudhya, C.; Tantimongcolwat, T.; Nantasenamat, C.; Galla, H. J. EDTA-induced Membrane Fluidization and Destabilization: Biophysical Studies on Artificial Lipid Membranes. Acta biochimica et biophysica Sinica, 2007, 39(11), 901-913.
[4] Managaro, A.; Wertz, P. The effect of permeabilizer on the in vitro penetration of propranolol through porcine buccal epithelium.
[5] Date, A. A.; Desai, N.; Dixit, R.; Nagarsenker, M. Self-nanoemulsifying Drug Delivery Systems: Formulation Insights, Applications and Advances. Nanomedicine, 2010, 5(10), 1595-1616.
[6] Pouton, C. W. Formation of poorly water-soluble drugs for oral administration: Physicochemical and physiological issues and the lipid formulation classification system. European Journal of Pharmaceutical Sciences, 2006, 29(3-4), 278-287.

SEQUENCE LISTING
SEQ ID NO: 1
MSMYFNKIISFNIISRIVICIFLICGMFMAGASEKYDANA
PQQVQPYSVSSSAFENLHPNNEMESSINPFSASDTERNAA
IIDRANKEQETEAVNKMISTGARLAASGRASDVAHSMVGD
AVNQEIKQWLNRFGTAQVNLNFDKNFSLKESSLDWLAPWY
DSASFLFFSQLGIRNKDSRNTLNLGVGIRTLENGWLYGLN
TFYDNDLTGHNHRIGLGAEAWTDYLQLAANGYFRLNGWHS
SRDFSDYKERPATGGDLRANAYLPALPQLGGKLMYEQYTG
ERVALFGKDNLQRNPYAVTAGINYTPVPLLTVGVDQRMGK
SSKHETQWNLQMNYRLGESFQSQLSPSAVAGTRLLAESRY
NLVDRNNNIVLEYQKQQVVKLTLSPATISGLPGQVYQVNA
QVQGASAVREIVWSDAELIAAGGTLTPLSTTQFNLVLPPY
KRTAQVSRVTDDLTANFYSLSALAVDHQGNRSNSFTLSVT
VQQPQLTLTAAVIGDGAPANGKTAITVEFTVADFEGKPLA
GQEVVITTNNGALPNKITEKTDANGVARIALTNTTDGVTV
VTAEVEGQRQSVDTHFVKGTIAADKSTLAAVPTSIIADGL
MASTITLELKDTYGDPQAGANVAFDTTLGNMGVITDHNDG
TYSAPLTSTTLGVATVTVKVDGAAFSVPSVTVNFTADPIP
DAGRSSFTVSTPDILADGTMSSTLSFVPVDKNGHFISGMQ
GLSFTQNGVPVSISPITEQPDSYTATVVGNSVGDVTITPQ
VDTLILSTLQKKISLFPVPTLTGILVNGQNFATDKGFPKT
IFKNATFQLQMDNDVANNTQYEWSSSFTPNVSVNDQGQVT
ITYQTYSEVAVTAKSKKFPSYSVSYRFYPNRWIYDGGRSL
VSSLEASRQCQGSDMSAVLESSRATNGTRAPDGTLWGEWG
SLTAYSSDWQSGEYWVKKTSTDFETMNMDTGALQPGPAYL
AFPLCALSI
SEQ ID NO: 2
AAVIGDGAPANGKTAITVEFTVADFEGKPLAGQEVVITTN
NGALPNKITEKTDANGVARIALTNTTDGVTVVTAEVEGQR
QSVDTHFVKGTIAADKSTLAAVPTSIIADGLMASTITLEL
KDTYGDPQAGANVAFDTTLGNMGVITDHNDGTYSAPLTST
TLGVATVTVKVDGAAFSVPSVTVNFTADPIPDAGRSSFTV
STPDILADGTMSSTLSFVPVDKNGHFISGMQGLSFTQNGV
PVSISPITEQPDSYTATVVGNSVGDVTITPQVDTLILSTL
QKKISLFPVPTLTGILVNGQNFATDKGFPKTIFKNATFQL
QMDNDVANNTQYEWSSSFTPNVSVNDQGQVTITYQTYSEV
AVTAKSKKFPSYSVSYRFYPNRWIYDGGRSLVSSLEASRQ
CQGSDMSAVLESSRATNGTRAPDGTLWGEWGSLTAYSSDW
QSGEYWVKKTSTDFETMNMDTGALQPGPAYLAFPLCALSI
SEQ ID NO: 3
MSLYRISSLHQAKQLNKNKQLNKTRISKSVVWANIVIQAT
FPLSTAFTPAVMAAETVGASDEKPRSASQAEQSTANAATR
LASILTNDDSAKQASSIARGTAANAGNEALQKWFNQFGSA
KVQLNLDEKLSLKGSQLDVLLPLTDSPDLLTFTQLGGRYI
DDRVTLNVGLGQRHFFAQQMLGYNLFVDHDASYSHTRIGV
GAEYGRDFINLAANGYFGVSGWKNSPDLDKYDEKVANGFD
LRSEAYLPTLPQLGGKLIYEQYFGDEVGLFGVDNRQKNPL
AVTLGVNYTPIPLFTVGVDHKMGRAGMNDTRFNLGFNYAF
GTPLAHQLDSDAVAIKRSLMGSRYNLVDRNNQIVMKYRKQ
NRVTLELPARVSGAARQTMPLVANATAQQGIDRIEWEASA
LTLAGGKITGSGNNWQITLPSYLSGGEGNNTYRISAIAYD
TLGNASPVAYSDLVVDSHGVNTNASGLTAAPEILPANASA
SSVIEFNIKDNANQPITGIADELAFSLELVELPEELAKAK
ARSVPLKTVSHTLTKITESAPGIYQATLTSGSKPQLINTT
AQINGVPLADVQTKVTLIADENTATLQTSSLQIITNGSLA
DDTDANQIRAVVVDAYGNKLSGVQVNFTVGNNAKITETTL
SDKQGGVTAAITSTKAGTYTVTAELNGVTQQIDVNFIPDA
GTATLDDSDEYKLQWVTNGQVADGESTNSVQLTVVDKFGN
TVPGVDVAFTTDIGAIISEVTPTDANGVATAKIISSQAKS
HTVKATLNRKEQTVEVNFIADTATAEITANNFTVEVDGQV
AGSGTNQVQALVVDKKGNPVANMTVNFTATNGVVAETTSA
KTDENGKVTTNLSMTNVGGTISTVTATMINSANVTSTQDK
PVIFYPDFTKATLNTPANTYSGFNINSGFPTTGFKNTHFQ
LSPHGITGANSDYDWVSSHPNVSVSNTGAITLQDNPGGKV
TITATWKHDSSKVFTYDFTLNYWVGLYSSTNLSWAQANAS
CINAGMRLPTNSEVSAGQDVRGVGSLFGEWGNLNAYPSFP
TAQIIWTSVDTNDFHIDTGLTHSASNVTLAYMCIK
SEQ ID NO: 4
MLNYFRAILISWKWKLSHHTSRPHDVKEKGHPRKIKVVAW
ITLFFQFAFPLSLSFTPAIAAANTTNSAPTSVITPVNASI
LPPAARATEPYTLGPGDSIQSIAKKYNITVDELKKLNAYR
TFSKPFASLTTGDEIEVPRKESSFFSNNPNENNKKDVDDL
LARNAMGAGKLLSNDNTSDAASNMARSAVTNEINASSQQW
LNQFGTARVQLNVDSDFKLDNSALDLLVPLKDSESSLLFT
QLGVRNKDSRNTVNIGAGIRQYQGDWMYGANTFFDNDLTG
KNRRVGVGAEVATDYLKFSANTYFGLTGWHQSRDFSSYDE
RPADGFDIRTEAYLPAYPQLGGKLMYEKYRGDEVALFGKD
DRQKDPHAVTLGVNYTPVPLVTIGAEHREGKGNNNNTSVN
VQLNYRMGQPWNDQIDQSAVAANRTLAGSRYDLVERNNNI
VLDYKKQELIHLVLPDRISGSGGGAITLTAQVRAKYGFSR
IEWDATPLENAGGSTSPLTQSSLSVTLPFYQHILRTSNTH
TISAVAYDAQGNASNRAVTSIEVTRPETMVISHLATTVDN
ATANGIAANTVQATVTDGDGQPIIGQIINFAVNTQATLST
TEARTGANGIASTTLTHTVAGVSAVSATLGSSSRSVNTTF
VADESTAEITAANLTVTTNDSVANGSDTNAVRAKVTDAYT
NAVANQSVIFSASNGATVIDQTVITNAEGIADSTLTNTTA
GVSAVTATLGSQSQQVDTTFKPGSTAAISLVKLADRAVAD
GIDQNEIQVVLRDGTGNAVPNVPMSIQADNGAIVVASTPN
TGVDGTINATFTNLRAGESVVSVTSPALVGMTMTMTFSAD
QRTAVVSTLAAIDNNAKADGTDTNVVRAWVVDANGNSVPG
VSVTFDAGNGAVLAQNPVVTDRNGYAENTLTNLAIGTTTV
KATTVTDPVGQTVNTHFVAGAVDTITLTVLVNGAVANGVN
TNSVQAVVSDSGGNPVNGAAVVFSSANATAQITTVIGTTG
VDGIATATLTNTVAGTSNVVATIDTVNANIDTTFVAGAVA
TITLTTLVNGAVADGANSNSVQAVVSDSGGNPVTGAAVVF
SSANATAQITTVIGTTGVDGIATATLTNTVAGTSNVVATI
GSITNNIDTAFVAGAVATITLTTPVNGAVADGANSNSVQA
VVTDSGGNPVNGAAVVFSSANATAQITTVIGTTGADGIAT
ATLTNTVAGTSNVVATVDTVNANIDTTFVAGAVATITLTT
PVNGAVADGADSNSVQAVVSDSGGNPVAGAAVVFSSANAT
AQVTTVIGTTGADGIATATLTNTVAGTSNVVATIGSITNN
IDTAFVAGAVATITLSVPVNDATADGVDTNQVDALVQDAN
GNATTGAAVVFSSTNGADIIVPTMNTGVNGVASTLLTHTV
AGTSNVVATVDTVNANIDTAFVPGAVATITLTTPVNGAVA
DGANSNSVQAVVSDSEGNAVAGAAVVFSSANATAQITTVI
GTTGADGIATATLTNTVAGTSNVVATIDTVNANIDTAFVP
GAVATITLSVLVNDATADGADTNQVDALVQDANGNATTGA
AVVFSSANGADIIAPTMNTGVNGVASTLLTHTQSGVSNVV
ATIDTVNANIDTTFVAGAVAAITLTTPVDGAVADGTDSNS
VQAVVSDSEGNAVAGAAVVFSSANATAQITTVIGTTGADG
IATATLTNTVAGTSNVAATIGSITDNIDTVFVAGAVATIT
LSVPVNDATADGADTNQVDALVQDVNGNATTGAAVVFSSA
NGATILSSTVNTGADGIASTTLTHTQSGVSNVVATIDTVN
ANIDTTFVAGAVATITLSVLVNDATADGADTNQVDALVQD
ANGNATTGAAVVFSSANGATIIVPTMNTGANGVASTLLTH
TVAGTSNVVATIGSITNNIDTAFVAGAVATITLTTPVNGA
VADGANSNSVQAVVSDSEGNAVAGAAVVFSSANATAQITT
VIGTTGADGIATATLTNTVAGTSNVVATIGSITDNIDTVF
VAGAVATITLTTPVNGAVADGANSNSVQAVVSDSEGNPVT
GATVVFSSSNATAQITTVIGTTGADGIATATLTNTVAGTS
NVVATIDTVNANIDTTFVPGAVATITLTTPVDGAVADGAN
SNSVQAVVTDSGGNPVTGAAVVFSSANATAQITTVIGTTG
ADGIATATLTNTVAGTSNVVATVDTVNANIDTTFVAGAVA
TITLTTPVNGAVANGADSNSVQAVVSDSEGNAVAGAAVVF
SSANATAQITTVIGTTGADGIATATLINTVAGTSNVVATI
DTVNANIDTTFVAGAVATITLTTPVDGAVANGADSNSVQA
VVSDSEGNAVAGAAVVFSSANATAQITTVIGTTGADGIAT
ATLTNTVAGTSNVVATIGSITNNIDTAFVAGAVATITLTT
PVNGAVADGANSNSVQAVVTDSGGNPVNGAAVVFSSANAT
AQITTVIGTTGADGIATATLTNTVAGTSNVVATVDTVNAN
IDTTFVAGAVATITLTTPVNGAVADGADSNSVQAVVSDSG
GNPVAGAAVVFSSANATAQVTTVIGTTGADGIATATLTNT
VAGTSNVVATIGSITNNIDTAFVAGAVATITLTTPVNGAV
ADGADSNSVQAVVSDSEGNAVTGAAVVFSSANATAQITTV
IGTTGADGIATATLTNTVAGTSNVVATIGGITNNIDTAFV
AGAVATITLTTPVNGAVADGTDSNSVQAVVSDSEGNAVAG
AAVVFSSANATAQITTVIGTTGADGIATATLTNTVAGTSN
VVATIGSITNNIDTAFVAGAVATITLTTLVNGAVANGADS
NSVQAVVSDSGGNVVAGATVVFSSTNATAQVTTVIGTTGA
DGIATATLTNTVAGTSNVVATIDTVNANIDTTFVAGAVAT
ITLSVLVNDATADGADTNQVDALVQDANGNATTGAAVVFS
SANGATILSSTMNTGVNGVASTLLTHTVAGTSNVVATIDT
VNANIDTAFVAGAVATITLTTPVNGAVANGADSNSVQAVV
SDSEGNAVAGAAVVFSSANATAQITTVIGTTGVDGIATAT
LTNTVAGTSNVVATVDTVNANIDTAFVAGAVATITLTTPV
NGAVANGADSNSVQAVVSDSGGNVVAGATVVFSSTNTTAQ
VTTVIGTTGADGIATATLTNTVAGTSNVVATVDTVNANID
TTFVAGAVATITLSVLVNDATADGADTNQVDALVQDANGN
ATTGAAVVFSSANGADIIAPTMNTGVNGVASTLLTHTMAG
TSNVIATIDTVNANIDTTFVAGAVATITLSVPVNDATADG
ADTNQVDALVQDANGNATTGAAVVFSSANGATILSSTMNT
GVNGVASTLLTHTQSGVSNVVATIDTVNANIDTAFVAGAV
ATITLTTPVNGAVADGANSNSVQAVVTDSGGNPVNGAAVV
FSSANATAQITTVIGTTGADGIATATLTNTVAGTSNVAAT
IDTVNANIDTTFVAGAVATITLTTPVNGAVADGANSNSVQ
AVVSDSEGNPVNGATVVFSSINATAQITTVIGTTGVDGIA
TATLTNTVAGTSNVVATIDTVNANIDTTFVAGAVATITLT
TLVNGAVADGANSNSVQAVVSDSGGNPVTGAAVVFSSANA
TAQITTVIGTTGVDGIATATLTNTVAGTSNVVATIGSITN
NIDTAFVAGAVATITLTTPVNGAVADGANSNSVQAVVTDS
GGNPVNGAAVVFSSANATAQITTVIGTTGADGIATATLTN
TVAGTSNVIATIDTVNANIDTTFVAGAVATITLTTPVNGA
VADGADSNSVQAVVSDSEGNAVTGAAVVFSSANATAQITT
VIGTTGADGIATATLTNTVAGTSNVVATIDTVNANIDTAF
VAGELENIVVSIINNNALANGADTNIVEAFVTDRFGNGVA
NQSLMFGTNGASIVGSSTVTTNIDGRVRVSATHTVAGSSN
TVFAISGAHQGYTRVTFVADASTAQLKLTSFLDNQLANGK
AGNIAQALVTDAYDNPLANQSVSFALDNGAVIESRGDASS
ASGIVLMRFNNTLAGMTTVTATLDSTGQTETLEMHFVAGK
AASIELTMTKDNAVANNIDTNEVQVLVTDADGNAINGAVV
NLTSNSGMNITPNSVTTGSDGTATATLTHTLAGSLPINAR
IDQVSKTINATFIADVSTAQIIASDMFIIVNDQVANGQAV
NAVQARVTDSYGNPIQGQLVEFVLSNTGTIQYKLEETSVE
GGVMVTFTNTLAGITNVTATVVSSRSSQNVDTTFIADVTT
AHIAESDLMVIVDNAVANNSEKNEVHARVTDAKGNVLSGQ
TVIFTSGNGAAITTVNGISDGDGLTKATLTHTLAGTSVVT
ARVGNQVQSKDTTFIADRTTATIRASDLTITRSNALADGV
ATNAARVIVTDAYGNPVPSMLVSYTSENGATLTPTLGSTD
SSGMLSTTFTHTIAGISKVTATIVTMGISQAKDAVFIADR
TTAHVSALTVEKNDSLANNSDRNIVQAHIQDAHGNVITGM
NVNFSATENVTLAANMVTTNAQGYAENTLRHNAPVTSAVT
ATVATDLVGLTEDVRFVAGAGARIELFRLNDGAVADGIQT
NRVEARVYDVSDNLVPNSNVVFSADNGGQLVQNDVQTDAL
GSAYVTVSNINTGVTKVSVTADGVSASTTTTFIADKDTVT
LRADLFLITHDNAVANGVTENRVLLQLLDANDNKVSGVEV
NFTATNGASINASAITDTNGLAIGVLTNTLSGPSDVTVTL
VTPGGTESLTVTPQFIADINTARIANGDFVIIDDGAVANS
VDANEVRARVTDNQGNAIAGYSVTFASQNGATITTSGITG
VDGWASAKLTHTKAGESGILARISRPGSMVQVLTPYFIAD
VSTATLQLFNFNPIPIIADGVMQFFVLGRVFDANQNPVGG
QQVAFSATNEVTLTESNGSISTPEGSVLLSVTSTQAGVHP
ITGTLVSNNYTDTFGATFIANKNTAQLSTLMVVDNNALAD
GVTRNQVRAHVVDSTGNSVADIAVTFTANHGAQLSHVTVL
TDDNGDAVNTLTNSLVGVTVVTAKLGTAGTPLTVDTVFTA
GPLATLTLVTMVDNAFADNSATNTVQATLKDATGNPIVGE
VVAFAASNGATITATDGGVSNANGIVLATLTNGAAGVSTV
TATIETLTATTETTFIAMKNLDVTVGDTTFDGDAGFPTTG
FVGAAFKVNSGGDNSLYDWSSSAPALVSVSGEGVVTFNAV
FPTGTPAITISATPKGGGSPLSYSFRVNQWFINNNGVALN
RADAATYCANAGYTTVSSSQVTNAIVWGMGTRAMGNLWSE
WGDFNNYNVPGWEPAEFFWLSDNYNATDGLAASLSHGVLT
TMGDPMAMIHVMCTRPI
SEQ ID NO: 5
MIKYFSFFKKPEPIVGILPNRQSHHILPTHIRRVAWGTLL
LQLFIPLSVSFSPAIAAMKASKADTMVSYSSTEPYVLGSG
ETVAMVAKKYGITVDELKKINIYRTFSRPFTALTTGDEID
IPRKASPFSVDNNKDNRLSVENTLAGHAVAGATALSNGDV
AKSGERMVRSAASNEFNNSAQQWLSQFGTARVQLNINDDF
HLDGSAADVLIPLYDNEKSILFTQLGARNKDSRNTVNMGA
GVRTFQGNWMYGANTFFDNDLTGKNRRIGVGAEAWTDYLK
LSANNYFGITDWHQSRDFIDYNERPANGYDLRAEAYLPSY
PQLGGKAMYEKYRGDDVALFGKDNRQKNPHAITAGVNYTP
IPLVTIGAEHRAGKGGQNDSNINFQLNYRLGETWQSHIDP
SAVAASRTLAGSRYDLVERNNHIVLDYQKQNLVRLSLPDS
LAGDPFSQLSVTAQVTATHGLERIDWQSAELMAAGGVLKQ
TSKNGLEITLPEYQMNRTGGNSYILNAIAYDTQGNASSQA
SMLITVNAQKINIANSTLVAVPINIEANNSDTSVVTLTLK
DDNNIPVTGQDVTFLSPLGTLSAMTDSGNGVYTATLTAGT
VSGTTAVSSNINGSALDMTPATVTLNGNSGELSITHSMLV
AAPVNIEANGSDTSVVTLTLRDSNNNPVTGQTVTFAGTLG
TLGAVTEGSSGVYTATLTAGIMVGTSSITASVNSTALGVT
PATVTLNGDSGNLSTTNSTLVAAPVNIEANSSDTSVVTLT
LRDNNNNPVTGQTVVFTSTLGTLGNVTEQASGVYTATLTA
GTVSGVASLSVSVGGNALGVTPATVTLNGDSGNLSTTNST
LVAAPVNIEANSSDTSVVTLTLRDNNNNPVTGQTVNFAGT
LGTLGTVSEGSSGVYTTTLTAGTVAGVASLSVNVGGNALG
VTPATVTLNGNSGNLSATNSTLVAAPVNIEANSSDTSVVT
LTLRDNNNNPVTGQTVAFTSTLGTLGNVTEQASGVYTATL
TAGTVSGVASLSVSVNSNALGVTPATVTLNGDSGNLSTTN
STLVAAPVNIEANSSDTSVVTLTLRDNNNNPVTGQTVAFT
STLGTLGNVTEQASGLYTATLTAGTVSGVASLSVNVGGNA
LGVTPATVTLNGDSGNLSATNSTLVAAPVNIEANSSDTSV
VTLTLRDNNNNPVTGQTVAFTSTLGTLGNVTEQASGLYTA
TLTAGTVSGVASLSVNVGGTALGVTPATVTLNGDSGNLST
TNSTLVAAPVNIEANSSDTSVVTLTLRDNNNNPVTGQTVA
FTSTLGTLGNVTEQASGLYTATLTAGTVSGVASLSVSVNS
TALGVTPATVTLNGDSGNLSTTNSTLVAAPVNIEANSSDT
SVVTLTLRDNNNNPVTGQTVAFTSTLGTLGNVTEQASGVY
TATLTAGTVAGVASLSVNVGGNALGVTPATVTLNGDSGNL
STTNSTLVAAPVNIEANSSDTSVVTLTLRDNNNNPVTGQT
VAFTSTLGTLGNVTEQASGVYTATLTAGTVSGVASLSVSV
GSSALGVTPATVTLNGDSGNLSTTNSTLVAAPVNIEANNS
DTSVVTLTLRDNNNNPVTGQTVAFTSTLGTLGNVTEQASG
VYTATLTAGTVSGVASLSVSVNSNALGVTPATVTLNGDSG
NLSTTNSTLVAAPVNIEANSSDTSVVTLTLRDNNNNPVTG
QTVVFTSTLGTLGNVTEQASGLYTATLTAGTVSGVASLSV
SVGGNALGVTGNITLAPGALDAARSILAVNKPSINADDRI
GSTITFTAQDAQGNAITGLDIAFMTDLENSQIMTLVDHND
GTYTANINGTQTGIANIAVQSSGATIAGLAATMVTITPGA
WNTTQATPVMTVALPITTCQSSSGVYKRYYIGIVTHELYD
NYGNEISGILTYNLGAGRYTTVTSQNSSVSGSNGLTRRSN
SEQ ID NO: 6
MYSFFNTLTVTKIISRLILSIGLIFGIFTYGFSQQHYFNS
EALENPAEHNEAFNKIISTGTSLAVSGNASNITRSMVNDA
ANQEVKHWLNRFGTTQVNVNFDKKFSLKESSLDWLLPWYD
SASYVFFSQLGIRNKDSRNTLNIGAGVRTFQQSWMYGFNT
FYDNDMTGHNHRIGVGAEAWTDYLQLSANGYFRLNGWHQS
RDFADYNERPASGGDIHVKAYLPALPQLGGKLKYEQYRGE
RVALFGKDNLQSNPYAVTTGLIYTPIPFITLGVDQRMGKS
RQHEIQWNLQMDYRLGESFRSQFSPAVVAGTRLLAESRYN
LVERNPNIVLEYQKQNTIKLAFSPAVLSGLPGQVYSVSAQ
IQSQSALQRILWNDAQWVAAGGKLIPVSATDYNVVLPPYK
PMAPASRTVGKTGESEAAVNTYTLSATAIDNHGNSSNPAT
LTVIVQQPQFVITSEVTDDGALADGRTPITVKFTVTNIDS
TPVAEQEGVITTSNGALPSKVTKKTDAQGVISIALTSFTV
GVSVVTLDIQGQQATVDVRFAVLPPDVTNSSFNVSPSDIV
ADGSMQSILTFVPRNKNNEFVSGITDLEFIQSGVPVTISS
VTENADNYTASVVGNSVGDVDITPQVGGESLDLLQKRITL
YPVPKITGIKVNGEQFATDKGFPKTTFNKATFQLVMNDDV
ANNTQYDWTSSYAASAPVDNQGKVNIAYKTYGSTVTVTAK
SKKFPSYTATYQFKPNLWVFSGTMSLQSSVEASRNCQRTD
FTALIESARASNGSRSPDGTLWGEWGSLATYDSAEWPSGN
YWTKKTSTDFVTMDMTTGDIPTSAATAYPLCAEPQ
SEQ ID NO: 7
MGSIFKGIERYLCAGFMKKAIAYTQIILQILLGTLPLYSM
SFSTQANSDITKKTVLFKQLHTLTPTDTLESVAASYGLSV
DELWALNINLYNNRSAFDAIKYGAVVYVPNQEEEQQAAQQ
ASLVASHLSQVGNSLSSENRVDAFSRLAKGILLSSTAKTV
EEWLGHIGQAQVKLQTDDKNDFSGSEIDLFIPLYDQPEKL
AFSQFGFRRIDQRNIMNIGLGQRHYVSDWMFGYNIFFDQQ
VSGNAHRRVGFGGELARDYIKLSANSYHRLGGWKNSTRLE
DYDERAANGYDIRTEAYLPHYPQLGGKLMYEQYFGDEVAL
FGINERQKNPSALTAGVSYTPIPLVSLGLDHTIGNGGKKK
TGVNVAVNYEINTPWQQQIDPAAVQTTRTLAGRRMDLVDR
NNNIVLEYRKQQVVTLNLPEKVSGKEKQVVPINYTFNARH
GLDRIEWDAADVIKAGGQVINQGNLAYYIAMPPYIDGAVN
AYVLSGRAIDKKGNYSVSGSTNVYVTGVNINRINSTISLN
PATLPANGTSRSTIQLKLNTDAGQAVSGASGQMTFATRDS
SGRVFKARTSLQPVVISDVQEVQTGVYEASITSGFLTGRF
EITPTVRGVQLNPIILTQSADKTTATITDSSAVTISTPSI
TTNATDKTKLEVQVTDALGHPVPGVEVTWVSDLNSPGLEY
VTSITNEHGIAENNFSSTVTGTANITVQVGTSAPVQAGKI
EIKADNSTMTVNASDFTVTTTPVVANGTSKAVYKLKVMDK
QGNVVPGAAVDWLSNIGTFVQGSTTTTDTNGETFIELVST
KAETAKVTATVGGKPYNAGKVVFVADRQSGKITLLPVSKN
TAAANGTDSITLNAKILDANGNPIKNEEIEWDAASHKVTF
SPATGKTQTNDLGETQITLTSTDVGDITLNAQVVKNNLLV
NQAGEKLSFTADTVTANISAWSAPSVKTLIADGQAQVIYK
VVVKDKNGHVVPNSPVLWETNLGEFVPAQATTTMTSTDSQ
GEATVVLASIKAGSATVKASVNANKDTSPTQVEFTADSST
ATTATTPVTKQVYVANGSEKVTYAVTVLDANNNPVKAEAI
NWKSENGHPVKVEPAPSQTDGQGKATVSIGSVKAGDTQIR
ATLGNNATAIADAITFEADRQTAVVKTVEVTGSKVTAPDG
TGSISYVTTVVDANGNPVSGMILSWGSNINNVANPSTTTD
INGQSSQTITGTQAGKVEISVALTSGNNATNPVKNSNNAE
FVAVTPVMANADLLLQPNLIIANGKQTATLKFTLRDANHN
PVSGLKQRIDVTQSVASHVTIGAVTETTVKGVYQAAITGM
KENSVDLTASVKGTNVRQTRTLTLQADNKTATLKTVTSNI
KTAKADGKESITYRAKVIDAQGNASLDNVSVGWRTTLGEL
AAITKTDTSGIATVTLTSKQAGSATVTAIVSSTSEMKAAP
VNFTAGGISITQSTASLSVKDLVADDVITTKLTVNIKDDN
GNPLTGKGSEISVTATGLAGLKLPTTFVEGPNGVYTATIT
GTKAGVGDIVTALAGKELAKQQLKVIADVQTAKIADIKPL
KSGSVSVGDKVTYQATLKDANDNLLGAGIPVHWSVNRDTL
MSGKLISLTNSAGVAEVEISRDLAGDALVTAAVGNNSLQA
TAVKFISGGVDISKSSMQLLQGNITADNLDIATIQVDIRD
SKGNPLPNLASQITTSPKKGEHGLKIETIANPSGDGYLVK
MKGTQAGNHTVTVSVAGKPLSAKVDMVLKGDATTAKIESV
KSSSPTFKADNVDTVTYTAKVVDANNNLLENIAVSWRLAQ
GEGQYQGQSYTGKTGVATTKLSASRLGTYKMEAQVRQQVK
AAAGVNSTAGDADPSQSDFVVDVASIDSSGNTKAKLTATL
KDKFGNLLSGQKVKLTDSNSLKKITLSANPMKDNGDGTYS
TEVTATAKGNTRFIARVNGVDLTQQPQIVIGNIIPQLSFA
KSKEATTYSRKVHKPLSLTGLPSSATLTAHWSSDNSDVAT
VNPLNGELTLLKAGVVNISVLTLPTDTYTSGTANYQLTVE
KADPGINFAVAKRDVKWMDSMSPQNFVLSNSDANQSDIKT
IWQTDSGKIATVDKGGLVTLVKPGTTNVTVSFVGDERFKY
GEASYELNVAKYKPTVSFANSLLTNKVSEKIYVQKPDEKL
STYAHLETKWSSSDNAIVEVANDASYMSPKGPGKARITLQ
VVGNDWYEEQSSSYEQEVYATPKVSIRETTAISNSVKKVN
ERVWSPVFTNDNFGVTVDNSQSKYERADSVKVILLDGTQE
LASKELGITTSSSFEFKPKPDWVGKSLKVKVVAKNDVRQE
NEVTLDHEVRVGTLEPIDIWQNAIFTRNYSLHNNDGSKRD
SCPIVNNLFYPNYARLNWRMQLVLNKDMLHPMQITKLESK
TSKHGINMTHIDSSTSEIFDSYDNKDDNRLINKCIKEKYG
TYKTYMDIKYAGREYKYEAINDLYWEGEGDDRESDKSSGF
KKVP
SEQ ID NO: 8
MTKDFKISVSAALISALESSPYAFAEEPEDGNDGIPRLSA
VQISPNVDPKLGVGLYPAKPILRQENPKLPPRGPQGPEKK
RARLAEAIQPQVLGGLDARAKGIHSTATGATAEAAKPAAV
AVGAGSIATGVNSVAIGPLSKALGDSAVTYGASSTAQKDG
VAIGARASASDTGVAVGFNSKVDAQNSVAIGHSSHVAADH
GYSTATGDLSKTDRENSVSIGHESLNRQLTHLAAGTKDND
AVNVAQLKKEMAETLENARKETLAQSNDVLDAAKKHSNSV
ARTTLETAEEHANKKSAEALVSAKVYADSNSSHTLKTANS
YTDVTVSSSTKKAISESNQYTDHKFSQLDNRLDKLDKRVD
KGLASSAALNSLFQPYGVGKVNFTAGVGGYRSSQALAIGS
GYRVNESVALKAGVAYAGSSNVMYNASFNIEW
SEQ ID NO: 9
MTKDFKISVSAALISALESSPYAFANNDEVHFTAVQISPN
ADPDSHVVIFQPAAEALGGTNALAKSIHSIAVGASAEAAK
QAAVAVGAGSIATGVNSVAIGPLSKALGDSAVTYGAASTA
QKDGVAIGARAFTSDTGVAVGENSKVDAKNSVAIGHSSHV
AVDHDYSTATGDRSKTDRKNSVSIGHESLNRQLTHLAAGT
KDTDAVNVAQLKKEIEKTQVNANKKSAEVLGIANNYTDSK
SAETLENARKEAFDLSNDALDMAKKHSNSVARTTLETAEE
HTNKKSAETLARANVYADSKSSHTLQTANSYTDVTVSNST
KKAIRESNQYTDHKFRQLDNRLDKLDTRVDKGLASSAALN
SLFQPYGVGKVNFTAGVGGYRSSQALAIGSGYRVNESVAL
KAGVAYAGSSDVMYNASFNIEW

1. A film suitable for administration to an oral cavity comprising:
(i) an alginate salt of a monovalent cation or a mixture of alginate salts containing at least one alginate salt of a monovalent cation; and
(ii) a carrier system comprising:
(a) a carrier,
(b) a pathogen entry protein or fragment thereof, which specifically binds to a molecule on the surface of a mammalian target cell of said pathogen and which is covalently linked to the surface of said carrier, and
(c) at least one active pharmaceutical ingredient (API) or pharmaceutically acceptable salt thereof.

2. The film according to claim 1, wherein the alginate salt of a monovalent cation (a) comprises from 25 to 35% by weight of β-D-mannuronate and/or from 65 to 75% by weight of α-L-guluronate, and (b) has a weight average molecular weight of from 30,000 g/mol to 90,000 g/mol.

3. The film according to claim 1, wherein said carrier is selected from the group consisting of: nanoparticles, preferably matrices of solid-lipid nanoparticles (SLN); polymer particles, preferably nanocapsules; and vesicles, preferably liposomes or other artificially-prepared spherical or non-spherical vesicles.

4. The film according to claim 3, wherein the carrier is a liposome, preferably wherein the liposome is unilamellar or multilamellar and/or the overall charge of the liposome is positive, neutral or negative.

5. The film according to claim 1, wherein the molecule on the surface of a mammalian target cell is a receptor protein, preferably a Pi-integrin receptor.

6. The film according to claim 1, wherein the pathogen entry protein is from a bacterium that sequesters in a non-phagocytic cell, preferably wherein said bacterium is (i) a Gram-negative bacterium, preferably Chlamydia, Coxiella burnetii, Ehrlichia, Rickettsia, Legionella, Salmonella, Shigella, or Yersinia, or (ii) a Gram-positive bacterium, preferably Mycobacterium leprae, or Mycobacterium tuberculosis.

7. The film according to claim 5, wherein the pathogen entry protein is selected from the group consisting of invasin, YadA, internalin and other inv-type and related adhesive bacterial outer membrane molecules.

8. The film according to claim 1, wherein:
(a) the covalent link between the carrier and the pathogen entry protein is direct or via a linker; and/or
(b) the pathogen entry protein is linked via its C-terminus, its N-terminus or a side chain of an amino acid of said pathogen entry protein, preferably its N-terminus.

9. (canceled)

10. The film according to claim 7, wherein the pathogen entry protein is an invasin, and preferably wherein the invasin has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or variants thereof with at least 70% amino acid sequence identity and which specifically bind to the extracellular domain of β_{ι}-integrin receptor.

11. The film according to claim 1, wherein the fragment of the pathogen entry protein consists or essentially consists of the extracellular domain of the pathogen entry protein.

12. The film according to claim 11, wherein the API or pharmaceutically acceptable salt thereof is selected from the group consisting of small molecules; proteins; nucleic acids, preferably siRNA; nucleotides, preferably polynucleotides.

13. The film according to claim 1, wherein the alginate salt of a monovalent cation is selected from a sodium alginate, a potassium alginate and an ammonium alginate, and is preferably a sodium alginate.

14. The film according to claim 1, wherein the film comprises from 25% to 99% by weight of the alginate salt of a monovalent cation or the mixture of alginate salts containing at least one alginate salt of a monovalent cation, from 0% to 20% by weight of water, and from 0.001% to 75% by weight of the carrier system,
preferably wherein the film comprises from 29% to 93% by weight of the alginate salt of a monovalent cation or the mixture of alginate salts containing at least one alginate salt of a monovalent cation, from 5% to 15% by weight of water, and from 0.15% to 50% by weight of the carrier system.

15. (canceled)

16. The film according to claim 1, wherein the film further comprises at least one plasticizer which is sorbitol, glycerol, xylitol, or a combination thereof, preferably wherein the film comprises both sorbitol and glycerol.

17. The film according to claim 16, wherein the film further comprises from 0% to 40% by weight of sorbitol, and from 0% to 40% by weight of glycerol.

18. The film according to claim 1, wherein the film further comprises at least one pharmaceutically acceptable solvent, buffering component, filler, taste-masking agent, flavouring agent, acidifying agent, basifying agent, permeation enhancer, self-emulsifying drug delivery system (SEDDS), such as a self-microemulsifying drug delivery system (SMEDDS) or a self-nanoemulsifying drug delivery system (SMEDDS), chelating agent, antioxidant, antimicrobial agent, and/or inorganic salt.

19-20. (canceled)

21. A method of treating a disease or condition selected from infectious disease, diabetes mellitus, insulinoma, metabolic syndrome and polycysic ovary syndrome in a human patient, wherein said method comprises administering at least one film according to claim 1 to a human patient.

22. (canceled)

23. The method according to claim 21, wherein the film is administered to the oral cavity of the human patient.

24. A method of manufacturing a film according to claim 1, said method comprising:
(a) covalently linking a pathogen entry protein or part thereof to a carrier either prior or after contacting the carrier with at least one API or a pharmaceutically acceptable salt thereof, to form a carrier system;
(b) either:
(i) mixing the carrier in water, and optionally subsequently adjusting the pH of the solution to the desired level by addition of an appropriate acid or base, preferably a concentrated acid, and preferably adjusting the pH of the solution to from 2 to 4;
(ii) optionally, mixing one or more excipients into the solution; and
(iii) adding the alginate salt of monovalent cation under suitable conditions to result in the formation of a viscous cast;
or alternatively:
(i) mixing the carrier in an oil phase;
(ii) premixing a surfactant and a cosolvent, and then adding this to the solution obtained;
(iii) optionally, adding one or more excipients, flavouring agents, buffering components, permeation enhancers, chelating agents, antioxidants and/or antimicrobial agents to water in (i) under mixing;
(iv) adding water, or the solution obtained in (iii), to the solution obtained in (ii) under stirring, preferably continuous stirring, and more preferably wherein the water or the solution obtained in (iii) is added in a dropwise fashion; and
(v) mixing the alginate salt of monovalent cation in the solution, until a lump free dispersion is achieved, and optionally adding further water to modulate the viscosity of the cast formed;
(c) adjusting the pH of the solution to the desired level by addition of an appropriate acid or base, preferably a diluted acid or alkali, and preferably adjusting the pH of the solution to from 3 to 5;
(d) optionally, sonicating the cast;
(e) leaving the cast to de-aerate;
(f) pouring the cast onto a surface and spreading the cast out to the desired thickness;
(g) drying the cast layer at a temperature of from −10 to 30° C. and a pressure of from 0.5 to 1 atm, until the residual water content of the film is from 0 to 20% by weight and a solid film is formed; and
(h) optionally, cutting the solid film into pieces of the desired size, further optionally placing these pieces into pouches, preferably wherein the pouches are made from PET-lined aluminium, sealing the pouches and further optionally, labelling them.

25. The method of claim 24, wherein the pathogen entry protein and/or at least one constituent of the carrier comprises an activatable group prior to covalent linking, preferably wherein the activatable group is activated with an activating reagent selected from the group consisting of: carbodiimides, preferably N,N′-diisopropylcarbodiimide (DIC), N,N′-dicyclohexylcarbodiimide (DCC) or N-(3-Dimethylaminopropyl)-N′-ethylcarbodiimide hydrochloride (EDC); succinimidylesters, preferably sulfosuccinimide, N-hydroxybenzotriazole or N-hydroxysuccinimid (NETS); triazine-based coupling reagents, preferably 4-(4,6-Dimethoxy-1,3,5-triazin-2-yl)-4-methylmorpholiniumchloride (DMTMM); maleidesters; glutaraldehyde; and phosphonium or uronium based coupling agents, preferably benzotriazol-1-yloxytris(dimethylamino)phosphonium hexafluorophosphate (BOP), 1-Cyano-2-ethoxy-2-oxoethylidenaminooxy)dimethylamino-morpholino-carbenium hexafluorophosphate (COMU), 3-(diethoxyphosphoryloxy)-1,2,3-benzotriazin-4(3H)-one (DEPBT), 1-[Bis(dimethylamino)methylene]-1H-1,2,3-triazolo[4,5-b]pyridinium 3-oxid hexafluorophosphate (HATU), 2-(1H-benzotriazol-1-yl)-1,1,3,3-tetramethyluronium hexafluorophosphate (HBTU), O-(1H-6-Chlorobenzotriazole-1-yl)-1,1,3,3-tetramethyluronium hexafluorophosphate (HCTU), benzotriazol-1-yl-oxytripyrrolidinophosphonium hexafluorophosphate (PyBOP), (7-Azabenzotriazol-1-yloxy)tripyrrolidinophosphonium hexafluorophosphate (PyAOP), (Ethyl cyano(hydroxyimino)acetato-O²)tri-1-pyrrolidinylphosphonium hexafluorophosphate (PyOxim) and O—(N-Succinimidyl)-1,1,3,3-tetramethyluronium tetrafluoroborate (TSTU).


SEQUENCE LISTING (9 sequences)

SEQ ID NO: 1 - protein, 969 aa, Yersinia pseudotuberculosis
  InvA YPK_2429 (1)..(969)
MSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNSVGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGRSLVSSLEASRQCQGSDMSAVLESSRATNGTRAPDGTLWGEWGSLTAYSSDWQSGEYWVKKTSTDFETMNMDTGALQPGPAYLAFPLCALSI

SEQ ID NO: 2 - protein, 480 aa, Yersinia pseudotuberculosis
  pAK01aa 490-987 for overexpression (1)..(480)
AAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNSVGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGRSLVSSLEASRQCQGSDMSAVLESSRATNGTRAPDGTLWGEWGSLTAYSSDWQSGEYWVKKTSTDFETMNMDTGALQPGPAYLAFPLCALSI

SEQ ID NO: 3 - protein, 1075 aa, Yersinia pseudotuberculosis
  InvB YPK_2513 (1)..(1075)
MSLYRISSLHQAKQLNKNKQLNKTRISKSVVWANIVIQAIFPLSIAFTPAVMAAETVGASDEKPRSASQAEQSTANAATRLASILTNDDSAKQASSIARGTAANAGNEALQKWFNQFGSAKVQLNLDEKLSLKGSQLDVLLPLTDSPDLLTFTQLGGRYIDDRVTLNVGLGQRHFFAQQMLGYNLFVDHDASYSHTRIGVGAEYGRDFINLAANGYFGVSGWKNSPDLDKYDEKVANGFDLRSEAYLPTLPQLGGKLIYEQYFGDEVGLFGVDNRQKNPLAVTLGVNYTPIPLFTVGVDHKMGRAGMNDTRFNLGFNYAFGTPLAHQLDSDAVAIKRSLMGSRYNLVDRNNQIVMKYRKQNRVTLELPARVSGAARQTMPLVANATAQQGIDRIEWEASALTLAGGKITGSGNNWQITLPSYLSGGEGNNTYRISAIAYDTLGNASPVAYSDLVVDSHGVNTNASGLTAAPEILPANASASSVIEFNIKDNANQPITGIADELAFSLELVELPEELAKAKARSVPLKTVSHTLTKITESAPGIYQATLTSGSKPQLINITAQINGVPLADVQTKVTLIADENTATLQTSSLQIITNGSLADDTDANQIRAVVVDAYGNKLSGVQVNFTVGNNAKITETTLSDKQGGVTAAITSTKAGTYTVTAELNGVTQQIDVNFIPDAGTATLDDSDEYKLQWVTNGQVADGESTNSVQLTVVDKFGNTVPGVDVAFTTDIGAIISEVTPTDANGVATAKIISSQAKSHTVKATLNRKEQTVEVNFIADTATAEITANNFTVEVDGQVAGSGTNQVQALVVDKKGNPVANMTVNFTATNGVVAETTSAKTDENGKVTTNLSMTNVGGTISTVTATMINSANVTSTQDKPVIFYPDFTKATLNTPANTYSGFNINSGFPTTGFKNTHFQLSPHGITGANSDYDWVSSHPNVSVSNTGAITLQDNPGGKVTITATWKHDSSKVFTYDFTLNYWVGLYSSTNLSWAQANASCINAGMRLPTNSEVSAGQDVRGVGSLFGEWGNLNAYPSFPTAQIIWTSVDTNDFHIDTGLTHSASNVTLAYMCIK

SEQ ID NO: 4 - protein, 5337 aa, Yersinia pseudotuberculosis
  InvC YPK_0145 (01)..(5337)
MLNYFRAILISWKWKLSHHTSRPHDVKEKGHPRKIKVVAWITLFFQFAFPLSLSFTPAIAAANTTNSAPTSVITPVNASILPPAARATEPYTLGPGDSIQSIAKKYNITVDELKKLNAYRTFSKPFASLTTGDEIEVPRKESSFFSNNPNENNKKDVDDLLARNAMGAGKLLSNDNTSDAASNMARSAVTNEINASSQQWLNQFGTARVQLNVDSDFKLDNSALDLLVPLKDSESSLLFTQLGVRNKDSRNTVNIGAGIRQYQGDWMYGANTFFDNDLTGKNRRVGVGAEVATDYLKFSANTYFGLTGWHQSRDFSSYDERPADGFDIRTEAYLPAYPQLGGKLMYEKYRGDEVALFGKDDRQKDPHAVTLGVNYTPVPLVTIGAEHREGKGNNNNTSVNVQLNYRMGQPWNDQIDQSAVAANRTLAGSRYDLVERNNNIVLDYKKQELIHLVLPDRISGSGGGAITLTAQVRAKYGFSRIEWDATPLENAGGSTSPLTQSSLSVTLPFYQHILRTSNTHTISAVAYDAQGNASNRAVTSIEVTRPETMVISHLATTVDNATANGIAANTVQATVTDGDGQPIIGQIINFAVNTQATLSTTEARTGANGIASTTLTHTVAGVSAVSATLGSSSRSVNTTFVADESTAEITAANLTVTTNDSVANGSDTNAVRAKVTDAYTNAVANQSVIFSASNGATVIDQTVITNAEGIADSTLTNTTAGVSAVTATLGSQSQQVDTTFKPGSTAAISLVKLADRAVADGIDQNEIQVVLRDGTGNAVPNVPMSIQADNGAIVVASTPNTGVDGTINATFTNLRAGESVVSVTSPALVGMTMTMTFSADQRTAVVSTLAAIDNNAKADGTDTNVVRAWVVDANGNSVPGVSVTFDAGNGAVLAQNPVVTDRNGYAENTLTNLAIGTTTVKATTVTDPVGQTVNTHFVAGAVDTITLTVLVNGAVANGVNTNSVQAVVSDSGGNPVNGAAVVFSSANATAQITTVIGTTGVDGIATATLTNTVAGTSNVVATIDTVNANIDTTFVAGAVATITLTTLVNGAVADGANSNSVQAVVSDSGGNPVTGAAVVFSSANATAQITTVIGTTGVDGIATATLTNTVAGTSNVVATIGSITNNIDTAFVAGAVATITLTTPVNGAVADGANSNSVQAVVTDSGGNPVNGAAVVFSSANATAQITTVIGTTGADGIATATLTNTVAGTSNVVATVDTVNANIDTTFVAGAVATITLTTPVNGAVADGADSNSVQAVVSDSGGNPVAGAAVVFSSANATAQVTTVIGTTGADGIATATLTNTVAGTSNVVATIGSITNNIDTAFVAGAVATITLSVPVNDATADGVDTNQVDALVQDANGNAITGAAVVFSSTNGADIIVPTMNTGVNGVASTLLTHTVAGTSNVVATVDTVNANIDTAFVPGAVATITLTTPVNGAVADGANSNSVQAVVSDSEGNAVAGAAVVFSSANATAQITTVIGTTGADGIATATLTNTVAGTSNVVATIDTVNANIDTAFVPGAVATITLSVLVNDATADGADTNQVDALVQDANGNAITGAAVVFSSANGADIIAPTMNTGVNGVASTLLTHTQSGVSNVVATIDTVNANIDTTFVAGAVAAITLTTPVDGAVADGTDSNSVQAVVSDSEGNAVAGAAVVFSSANATAQITTVIGTTGADGIATATLTNTVAGTSNVAATIGSITDNIDTVFVAGAVATITLSVPVNDATADGADTNQVDALVQDVNGNAITGAAVVFSSANGATILSSTVNTGADGIASTTLTHTQSGVSNVVATIDTVNANIDTTFVAGAVATITLSVLVNDATADGADTNQVDALVQDANGNAITGAAVVFSSANGATIIVPTMNTGANGVASTLLTHTVAGTSNVVATIGSITNNIDTAFVAGAVATITLTTPVNGAVADGANSNSVQAVVSDSEGNAVAGAAVVFSSANATAQITTVIGTTGADGIATATLTNTVAGTSNVVATIGSITDNIDTVFVAGAVATITLTTPVNGAVADGANSNSVQAVVSDSEGNPVTGATVVFSSSNATAQITTVIGTTGADGIATATLTNTVAGTSNVVATIDTVNANIDTTFVPGAVATITLTTPVDGAVADGANSNSVQAVVTDSGGNPVTGAAVVFSSANATAQITTVIGTTGADGIATATLTNTVAGTSNVVATVDTVNANIDTTFVAGAVATITLTTPVNGAVANGADSNSVQAVVSDSEGNAVAGAAVVFSSANATAQITTVIGTTGADGIATATLINTVAGTSNVVATIDTVNANIDTTFVAGAVATITLTTPVDGAVANGADSNSVQAVVSDSEGNAVAGAAVVFSSANATAQITTVIGTTGADGIATATLTNTVAGTSNVVATIGSITNNIDTAFVAGAVATITLTTPVNGAVADGANSNSVQAVVTDSGGNPVNGAAVVFSSANATAQITTVIGTTGADGIATATLTNTVAGTSNVVATVDTVNANIDTTFVAGAVATITLTTPVNGAVADGADSNSVQAVVSDSGGNPVAGAAVVFSSANATAQVTTVIGTTGADGIATATLTNTVAGTSNVVATIGSITNNIDTAFVAGAVATITLTTPVNGAVADGADSNSVQAVVSDSEGNAVTGAAVVFSSANATAQITTVIGTTGADGIATATLTNTVAGTSNVVATIGGITNNIDTAFVAGAVATITLTTPVNGAVADGTDSNSVQAVVSDSEGNAVAGAAVVFSSANATAQITTVIGTTGADGIATATLTNTVAGTSNVVATIGSITNNIDTAFVAGAVATITLTTLVNGAVANGADSNSVQAVVSDSGGNVVAGATVVFSSTNATAQVTTVIGTTGADGIATATLTNTVAGTSNVVATIDTVNANIDTTFVAGAVATITLSVLVNDATADGADTNQVDALVQDANGNAITGAAVVFSSANGATILSSTMNTGVNGVASTLLTHTVAGTSNVVATIDTVNANIDTAFVAGAVATITLTTPVNGAVANGADSNSVQAVVSDSEGNAVAGAAVVFSSANATAQITTVIGTTGVDGIATATLTNTVAGTSNVVATVDTVNANIDTAFVAGAVATITLTTPVNGAVANGADSNSVQAVVSDSGGNVVAGATVVFSSTNTTAQVTTVIGTTGADGIATATLTNTVAGTSNVVATVDTVNANIDTTFVAGAVATITLSVLVNDATADGADTNQVDALVQDANGNAITGAAVVFSSANGADIIAPTMNTGVNGVASTLLTHTMAGTSNVIATIDTVNANIDTTFVAGAVATITLSVPVNDATADGADTNQVDALVQDANGNAITGAAVVFSSANGATILSSTMNTGVNGVASTLLTHTQSGVSNVVATIDTVNANIDTAFVAGAVATITLTTPVNGAVADGANSNSVQAVVTDSGGNPVNGAAVVFSSANATAQITTVIGTTGADGIATATLTNTVAGTSNVAATIDTVNANIDTTFVAGAVATITLTTPVNGAVADGANSNSVQAVVSDSEGNPVNGATVVFSSINATAQITTVIGTTGVDGIATATLTNTVAGTSNVVATIDTVNANIDTTFVAGAVATITLTTLVNGAVADGANSNSVQAVVSDSGGNPVTGAAVVFSSANATAQITTVIGTTGVDGIATATLTNTVAGTSNVVATIGSITNNIDTAFVAGAVATITLTTPVNGAVADGANSNSVQAVVTDSGGNPVNGAAVVFSSANATAQITTVIGTTGADGIATATLTNTVAGTSNVIATIDTVNANIDTTFVAGAVATITLTTPVNGAVADGADSNSVQAVVSDSEGNAVTGAAVVFSSANATAQITTVIGTTGADGIATATLTNTVAGTSNVVATIDTVNANIDTAFVAGELENIVVSIINNNALANGADTNIVEAFVTDRFGNGVANQSLMFGTNGASIVGSSTVTTNIDGRVRVSATHTVAGSSNTVFAISGAHQGYTRVTFVADASTAQLKLTSFLDNQLANGKAGNIAQALVTDAYDNPLANQSVSFALDNGAVIESRGDASSASGIVLMRFNNTLAGMTTVTATLDSTGQTETLEMHFVAGKAASIELTMTKDNAVANNIDTNEVQVLVTDADGNAINGAVVNLTSNSGMNITPNSVTTGSDGTATATLTHTLAGSLPINARIDQVSKTINATFIADVSTAQIIASDMFIIVNDQVANGQAVNAVQARVTDSYGNPIQGQLVEFVLSNTGTIQYKLEETSVEGGVMVTFTNTLAGITNVTATVVSSRSSQNVDTTFIADVTTAHIAESDLMVIVDNAVANNSEKNEVHARVTDAKGNVLSGQTVIFTSGNGAAITTVNGISDGDGLTKATLTHTLAGTSVVTARVGNQVQSKDTTFIADRTTATIRASDLTITRSNALADGVATNAARVIVTDAYGNPVPSMLVSYTSENGATLTPTLGSTDSSGMLSTTFTHTIAGISKVTATIVTMGISQAKDAVFIADRTTAHVSALTVEKNDSLANNSDRNIVQAHIQDAHGNVITGMNVNFSATENVTLAANMVTTNAQGYAENTLRHNAPVTSAVTATVATDLVGLTEDVRFVAGAGARIELFRLNDGAVADGIQTNRVEARVYDVSDNLVPNSNVVFSADNGGQLVQNDVQTDALGSAYVTVSNINTGVTKVSVTADGVSASTTTTFIADKDTVTLRADLFLITHDNAVANGVTENRVLLQLLDANDNKVSGVEVNFTATNGASINASAITDTNGLAIGVLTNTLSGPSDVTVTLVTPGGTESLTVTPQFIADINTARIANGDFVIIDDGAVANSVDANEVRARVTDNQGNAIAGYSVTFASQNGATITTSGITGVDGWASAKLTHTKAGESGILARISRPGSMVQVLTPYFIADVSTATLQLFNFNPIPIIADGVMQFFVLGRVFDANQNPVGGQQVAFSATNEVTLTESNGSISTPEGSVLLSVTSTQAGVHPITGTLVSNNYTDTFGATFIANKNTAQLSTLMVVDNNALADGVTRNQVRAHVVDSTGNSVADIAVTFTANHGAQLSHVTVLTDDNGDAVNTLTNSLVGVTVVTAKLGTAGTPLTVDTVFTAGPLATLTLVTMVDNAFADNSATNTVQATLKDATGNPIVGEVVAFAASNGATITATDGGVSNANGIVLATLTNGAAGVSTVTATIETLTATTETTFIAMKNLDVTVGDTTFDGDAGFPTTGFVGAAFKVNSGGDNSLYDWSSSAPALVSVSGEGVVTFNAVFPTGTPAITISATPKGGGSPLSYSFRVNQWFINNNGVALNRADAATYCANAGYTTVSSSQVTNAIVWGMGTRAMGNLWSEWGDFNNYNVPGWEPAEFFWLSDNYNATDGLAASLSHGVLTTMGDPMAMIHVMCTRPI

SEQ ID NO: 5 - protein, 1920 aa, Yersinia pseudotuberculosis
  InvD YPK_1315 (1)..(1920)
MIKYFSFFKKPEPIVGILPNRQSHHILPTHIRRVAWGTLLLQLFIPLSVSFSPAIAAMKASKADTMVSYSSTEPYVLGSGETVAMVAKKYGITVDELKKINIYRTFSRPFTALTTGDEIDIPRKASPFSVDNNKDNRLSVENTLAGHAVAGATALSNGDVAKSGERMVRSAASNEFNNSAQQWLSQFGTARVQLNINDDFHLDGSAADVLIPLYDNEKSILFTQLGARNKDSRNTVNMGAGVRTFQGNWMYGANTFFDNDLTGKNRRIGVGAEAWTDYLKLSANNYFGITDWHQSRDFIDYNERPANGYDLRAEAYLPSYPQLGGKAMYEKYRGDDVALFGKDNRQKNPHAITAGVNYTPIPLVTIGAEHRAGKGGQNDSNINFQLNYRLGETWQSHIDPSAVAASRTLAGSRYDLVERNNHIVLDYQKQNLVRLSLPDSLAGDPFSQLSVTAQVTATHGLERIDWQSAELMAAGGVLKQTSKNGLEITLPEYQMNRTGGNSYILNAIAYDTQGNASSQASMLITVNAQKINIANSTLVAVPINIEANNSDTSVVTLTLKDDNNIPVTGQDVTFLSPLGTLSAMTDSGNGVYTATLTAGTVSGTTAVSSNINGSALDMTPATVTLNGNSGELSITHSMLVAAPVNIEANGSDTSVVTLTLRDSNNNPVTGQTVTFAGTLGTLGAVTEGSSGVYTATLTAGIMVGTSSITASVNSTALGVTPATVTLNGDSGNLSTTNSTLVAAPVNIEANSSDTSVVTLTLRDNNNNPVTGQTVVFTSTLGTLGNVTEQASGVYTATLTAGTVSGVASLSVSVGGNALGVTPATVTLNGDSGNLSTTNSTLVAAPVNIEANSSDTSVVTLTLRDNNNNPVTGQTVNFAGTLGTLGTVSEGSSGVYTTTLTAGTVAGVASLSVNVGGNALGVTPATVTLNGNSGNLSATNSTLVAAPVNIEANSSDTSVVTLTLRDNNNNPVTGQTVAFTSTLGTLGNVTEQASGVYTATLTAGTVSGVASLSVSVNSNALGVTPATVTLNGDSGNLSTTNSTLVAAPVNIEANSSDTSVVTLTLRDNNNNPVTGQTVAFTSTLGTLGNVTEQASGLYTATLTAGTVSGVASLSVNVGGNALGVTPATVTLNGDSGNLSATNSTLVAAPVNIEANSSDTSVVTLTLRDNNNNPVTGQTVAFTSTLGTLGNVTEQASGLYTATLTAGTVSGVASLSVNVGGTALGVTPATVTLNGDSGNLSTTNSTLVAAPVNIEANSSDTSVVTLTLRDNNNNPVTGQTVAFTSTLGTLGNVTEQASGLYTATLTAGTVSGVASLSVSVNSTALGVTPATVTLNGDSGNLSTTNSTLVAAPVNIEANSSDTSVVTLTLRDNNNNPVTGQTVAFTSTLGTLGNVTEQASGVYTATLTAGTVAGVASLSVNVGGNALGVTPATVTLNGDSGNLSTTNSTLVAAPVNIEANSSDTSVVTLTLRDNNNNPVTGQTVAFTSTLGTLGNVTEQASGVYTATLTAGTVSGVASLSVSVGSSALGVTPATVTLNGDSGNLSTTNSTLVAAPVNIEANNSDTSVVTLTLRDNNNNPVTGQTVAFTSTLGTLGNVTEQASGVYTATLTAGTVSGVASLSVSVNSNALGVTPATVTLNGDSGNLSTTNSTLVAAPVNIEANSSDTSVVTLTLRDNNNNPVTGQTVVFTSTLGTLGNVTEQASGLYTATLTAGTVSGVASLSVSVGGNALGVTGNITLAPGALDAARSILAVNKPSINADDRIGSTITFTAQDAQGNAITGLDIAFMTDLENSQIMTLVDHNDGTYTANINGTQTGIANIAVQSSGATIAGLAATMVTITPGAWNTTQATPVMTVALPITTCQSSSGVYKRYYIGIVTHELYDNYGNEISGILTYNLGAGRYTTVTSQNSSVSGSNGLTRRSN

SEQ ID NO: 6 - protein, 835 aa, Yersinia enterocolitica
  InvA YE2564 (1)..(835)
MYSFFNTLTVTKIISRLILSIGLIFGIFTYGFSQQHYFNSEALENPAEHNEAFNKIISTGTSLAVSGNASNITRSMVNDAANQEVKHWLNRFGTTQVNVNFDKKFSLKESSLDWLLPWYDSASYVFFSQLGIRNKDSRNTLNIGAGVRTFQQSWMYGFNTFYDNDMTGHNHRIGVGAEAWTDYLQLSANGYFRLNGWHQSRDFADYNERPASGGDIHVKAYLPALPQLGGKLKYEQYRGERVALFGKDNLQSNPYAVTTGLIYTPIPFITLGVDQRMGKSRQHEIQWNLQMDYRLGESFRSQFSPAVVAGTRLLAESRYNLVERNPNIVLEYQKQNTIKLAFSPAVLSGLPGQVYSVSAQIQSQSALQRILWNDAQWVAAGGKLIPVSATDYNVVLPPYKPMAPASRTVGKTGESEAAVNTYTLSATAIDNHGNSSNPATLTVIVQQPQFVITSEVTDDGALADGRTPITVKFTVTNIDSTPVAEQEGVITTSNGALPSKVTKKTDAQGVISIALTSFTVGVSVVTLDIQGQQATVDVRFAVLPPDVTNSSFNVSPSDIVADGSMQSILTFVPRNKNNEFVSGITDLEFIQSGVPVTISSVTENADNYTASVVGNSVGDVDITPQVGGESLDLLQKRITLYPVPKITGIKVNGEQFATDKGFPKTTFNKATFQLVMNDDVANNTQYDWTSSYAASAPVDNQGKVNIAYKTYGSTVTVTAKSKKFPSYTATYQFKPNLWVFSGTMSLQSSVEASRNCQRTDFTALIESARASNGSRSPDGTLWGEWGSLATYDSAEWPSGNYWTKKTSTDFVTMDMTTGDIPTSAATAYPLCAEPQ

SEQ ID NO: 7 - protein, 2484 aa, Yersinia enterocolitica
  InvE YE0694 (01)..(2484)
MGSIFKGIERYLCAGFMKKAIAYTQIILQILLGTLPLYSMSFSTQANSDITKKTVLFKQLHTLTPTDTLESVAASYGLSVDELWALNINLYNNRSAFDAIKYGAVVYVPNQEEEQQAAQQASLVASHLSQVGNSLSSENRVDAFSRLAKGILLSSTAKTVEEWLGHIGQAQVKLQTDDKNDFSGSEIDLFIPLYDQPEKLAFSQFGFRRIDQRNIMNIGLGQRHYVSDWMFGYNIFFDQQVSGNAHRRVGFGGELARDYIKLSANSYHRLGGWKNSTRLEDYDERAANGYDIRTEAYLPHYPQLGGKLMYEQYFGDEVALFGINERQKNPSALTAGVSYTPIPLVSLGLDHTIGNGGKKKTGVNVAVNYEINTPWQQQIDPAAVQTTRTLAGRRMDLVDRNNNIVLEYRKQQVVTLNLPEKVSGKEKQVVPINYTFNARHGLDRIEWDAADVIKAGGQVINQGNLAYYIAMPPYIDGAVNAYVLSGRAIDKKGNYSVSGSTNVYVTGVNINRINSTISLNPATLPANGTSRSTIQLKLNTDAGQAVSGASGQMTFAIRDSSGRVFKARTSLQPVVISDVQEVQTGVYEASITSGFLTGRFEITPTVRGVQLNPIILTQSADKTTATITDSSAVTISTPSITTNATDKTKLEVQVTDALGHPVPGVEVTWVSDLNSPGLEYVTSITNEHGIAENNFSSTVTGTANITVQVGTSAPVQAGKIEIKADNSTMTVNASDFTVTTTPVVANGTSKAVYKLKVMDKQGNVVPGAAVDWLSNIGTFVQGSTTTTDTNGETFIELVSTKAETAKVTATVGGKPYNAGKVVFVADRQSGKITLLPVSKNTAAANGTDSITLNAKILDANGNPIKNEEIEWDAASHKVTFSPATGKTQTNDLGETQITLTSTDVGDITLNAQVVKNNLLVNQAGEKLSFTADTVTANISAWSAPSVKTLIADGQAQVIYKVVVKDKNGHVVPNSPVLWETNLGEFVPAQATTTMTSTDSQGEATVVLASIKAGSATVKASVNANKDTSPTQVEFTADSSTATIAITPVTKQVYVANGSEKVTYAVTVLDANNNPVKAEAINWKSENGHPVKVEPAPSQTDGQGKATVSIGSVKAGDTQIRATLGNNATAIADAITFEADRQTAVVKTVEVTGSKVTAPDGTGSISYVTTVVDANGNPVSGMILSWGSNINNVANPSTTTDINGQSSQTITGTQAGKVEISVALTSGNNATNPVKNSNNAEFVAVTPVMANADLLLQPNLIIANGKQTATLKFTLRDANHNPVSGLKQRIDVTQSVASHVTIGAVTETTVKGVYQAAITGMKENSVDLTASVKGTNVRQTRTLTLQADNKTATLKTVTSNIKTAKADGKESITYRAKVIDAQGNASLDNVSVGWRTTLGELAAITKTDTSGIATVTLTSKQAGSATVTAIVSSTSEMKAAPVNFTAGGISITQSTASLSVKDLVADDVITTKLTVNIKDDNGNPLTGKGSEISVTATGLAGLKLPTTFVEGPNGVYTATITGTKAGVGDIVTALAGKELAKQQLKVIADVQTAKIADIKPLKSGSVSVGDKVTYQATLKDANDNLLGAGIPVHWSVNRDTLMSGKLISLTNSAGVAEVEISRDLAGDALVTAAVGNNSLQATAVKFISGGVDISKSSMQLLQGNITADNLDIATIQVDIRDSKGNPLPNLASQITTSPKKGEHGLKIETIANPSGDGYLVKMKGTQAGNHTVTVSVAGKPLSAKVDMVLKGDATTAKIESVKSSSPTFKADNVDTVTYTAKVVDANNNLLENIAVSWRLAQGEGQYQGQSYTGKTGVATTKLSASRLGTYKMEAQVRQQVKAAAGVNSTAGDADPSQSDFVVDVASIDSSGNTKAKLTATLKDKFGNLLSGQKVKLTDSNSLKKITLSANPMKDNGDGTYSTEVTATAKGNTRFIARVNGVDLTQQPQIVIGNIIPQLSFAKSKEATTYSRKVHKPLSLTGLPSSATLTAHWSSDNSDVATVNPLNGELTLLKAGVVNISVLTLPTDTYTSGTANYQLTVEKADPGINFAVAKRDVKWMDSMSPQNFVLSNSDANQSDIKTIWQTDSGKIATVDKGGLVTLVKPGTTNVTVSFVGDERFKYGEASYELNVAKYKPTVSFANSLLTNKVSEKIYVQKPDEKLSTYAHLETKWSSSDNAIVEVANDASYMSPKGPGKARITLQVVGNDWYEEQSSSYEQEVYATPKVSIRETTAISNSVKKVNERVWSPVFTNDNFGVTVDNSQSKYERADSVKVILLDGTQELASKELGITTSSSFEFKPKPDWVGKSLKVKVVAKNDVRQENEVTLDHEVRVGTLEPIDIWQNAIFTRNYSLHNNDGSKRDSCPIVNNLFYPNYARLNWRMQLVLNKDMLHPMQITKLESKTSKHGINMTHIDSSTSEIFDSYDNKDDNRLINKCIKEKYGTYKTYMDIKYAGREYKYEAINDLYWEGEGDDRESDKSSGFKKVP

SEQ ID NO: 8 - protein, 432 aa, Yersinia pseudotuberculosis
  YadA pYV0013 (01)..(432)
MTKDFKISVSAALISALFSSPYAFAEEPEDGNDGIPRLSAVQISPNVDPKLGVGLYPAKPILRQENPKLPPRGPQGPEKKRARLAEAIQPQVLGGLDARAKGIHSIAIGATAEAAKPAAVAVGAGSIATGVNSVAIGPLSKALGDSAVTYGASSTAQKDGVAIGARASASDTGVAVGFNSKVDAQNSVAIGHSSHVAADHGYSIAIGDLSKTDRENSVSIGHESLNRQLTHLAAGTKDNDAVNVAQLKKEMAETLENARKETLAQSNDVLDAAKKHSNSVARTTLETAEEHANKKSAEALVSAKVYADSNSSHTLKTANSYTDVTVSSSTKKAISESNQYTDHKFSQLDNRLDKLDKRVDKGLASSAALNSLFQPYGVGKVNFTAGVGGYRSSQALAIGSGYRVNESVALKAGVAYAGSSNVMYNASFNIEW

SEQ ID NO: 9 - protein, 422 aa, Yersinia enterocolitica
  YadA Yep0066 (1)..(422)
MTKDFKISVSAALISALFSSPYAFANNDEVHFTAVQISPNADPDSHVVIFQPAAEALGGTNALAKSIHSIAVGASAEAAKQAAVAVGAGSIATGVNSVAIGPLSKALGDSAVTYGAASTAQKDGVAIGARAFTSDTGVAVGFNSKVDAKNSVAIGHSSHVAVDHDYSIAIGDRSKTDRKNSVSIGHESLNRQLTHLAAGTKDTDAVNVAQLKKEIEKTQVNANKKSAEVLGIANNYTDSKSAETLENARKEAFDLSNDALDMAKKHSNSVARTTLETAEEHTNKKSAETLARANVYADSKSSHTLQTANSYTDVTVSNSTKKAIRESNQYTDHKFRQLDNRLDKLDTRVDKGLASSAALNSLFQPYGVGKVNFTAGVGGYRSSQALAIGSGYRVNESVALKAGVAYAGSSDVMYNASFNIEW